(19) United States
(12) Patent Application Publication
(10) Pub. No.: US 2026/0331228 A1
(43) Pub. Date: Oct. 22, 2026

(54) TECHNOLOGIES FOR AUTOMATED CHEMICAL SIMULATIONS

(71) Applicant: Alexander G. Narinsky, Newton, MA (US)

(72) Inventors: Alexander G. Narinsky, Newton, MA (US); Boris Galitsky, Los Banos, CA (US); Artur Asatryan, Yerevan (AM)

(73) Assignee: Alexander G. Narinsky, Newton, MA (US)

(21) Appl. No.: 19/415,136

(22) Filed: Dec. 10, 2025

(51) Int. Cl. G16C 20/10

(52) U.S. Cl. CPC G16C 20/10

(57) ABSTRACT

A system and method are provided for automating chemical engineering process design and simulation using a hybrid neuro-symbolic approach and cloud-native infrastructure. A graphical user interface (GUI) enables users to construct and configure chemical process simulations by arranging process modules and specifying parameters. The system transforms the user-constructed process diagram into a structured, machine-readable graph model, which is then converted into infrastructure-as-code (IaC) artifacts for automated deployment of simulation components in a cloud environment. The deployed components execute the simulation, with results and feedback provided to the user via the GUI. In certain embodiments, artificial intelligence (AI), including large language models (LLMs) and neuro-symbolic reasoning, are employed to interpret process diagrams, validate configurations, select or configure computational models, and iteratively optimize process parameters based on simulation outcomes. The system supports both conventional and AI-augmented workflows, enabling scalable, reproducible, and explainable chemical process simulation and design.

### BACKGROUND

The field of chemical engineering simulation has traditionally relied on monolithic, first-principles models and proprietary software tools that are often limited in scalability, adaptability, and integration with modern computational infrastructure. As chemical processes become increasingly complex and the demand for high-throughput simulation, real-time optimization, and flexible design-space exploration grows, these conventional approaches struggle to keep pace. Existing solutions are typically bound to rigid graphical user interfaces or static file formats, making it difficult to automate workflows, integrate with cloud-native services, or adapt to evolving process requirements.

### BRIEF DESCRIPTION OF THE DRAWINGS

FIG. 1 is a simplified block diagram of a system for performing chemical simulations in a cloud environment.

FIG. 2 is a simplified block diagram of at least one embodiment of a compute device for performing chemical simulations.

FIG. 3 is a simplified block diagram of at least one embodiment of an environment that may be established by a compute device of FIG. 1.

FIG. 4 is a simplified view of one aspect of a graphical user interface for a chemical engineering simulation.

FIG. 5 is a simplified view of one aspect of a graphical user interface for a chemical engineering simulation.

FIG. 6 is a simplified flow diagram of one embodiment of a method for automating chemical engineering simulations using cloud-native infrastructure.

FIG. 7 is a simplified flow diagram of one embodiment of a method for automating chemical process simulation and design using a hybrid neuro-symbolic multi-agent system.

FIG. 8 is a data flow for one embodiment of a cloud-based chemical engineering simulation system.

### DETAILED DESCRIPTION

Industrial chemical process simulation has historically relied on deterministic, first-principles models implemented within large-scale, monolithic software environments. While such platforms (e.g., Aspen Plus™, gPROMS™, CHEMCAD™) have been widely adopted across the chemical, petrochemical, and pharmaceutical industries, several critical limitations hinder their applicability to modern process development and operation.

First, existing systems exhibit limited scalability in handling high-dimensional, nonlinear, and multi-phase systems. Traditional equation-oriented solvers often struggle with convergence under large process networks, recycle loops, or systems involving strong coupling between thermodynamics and reaction kinetics. This restricts their use in highly complex or integrated plant-wide simulations.

Second, current platforms are inflexible in integration with modern computational ecosystems. They are primarily designed for stand-alone execution with proprietary interfaces, which impedes interoperability with advanced optimization algorithms, cloud computing environments, real-time control systems, and machine learning frameworks. As a result, the ability to leverage high-performance computing or distributed simulation resources is constrained.

Third, many industrial simulation tools are static in their modeling paradigms. They emphasize steady-state or simplified dynamic modeling, which is insufficient for modern needs such as real-time digital twins, predictive maintenance, or adaptive process control. Incorporating uncertainty, stochastic behavior, or data-driven model augmentation is cumbersome, often requiring ad hoc customization.

Fourth, usability and accessibility limitations persist. Existing simulation environments typically rely on rigid graphical user interfaces or static input decks, limiting the ability to automate large-scale parametric studies, multi-objective optimization, or continuous model updating from process data. This restricts their utility in agile process development environments where rapid iteration is critical.

Fifth, there is a lack of standardized approaches to knowledge representation and modularization. Model libraries are often proprietary, incompatible across vendors, and difficult to extend with custom unit operations or new physical property packages. This hinders knowledge transfer, reusability, and collaboration across organizational or disciplinary boundaries.

Finally, industrial simulation systems face barriers to real-time deployment. High computational cost, long solution times, and poor numerical robustness prevent their use in online monitoring, closed-loop control, and operator training simulators that demand both speed and reliability.

Accordingly, there remains a need for improved methods and systems for chemical process simulation that (i) scale efficiently to complex systems, (ii) integrate seamlessly with modern computational and data environments, (iii) support real-time and data-driven applications, (iv) enable modular and reusable model development, and (v) offer greater adaptability and accessibility to a diverse range of users and use cases.

Modern chemical engineering simulation platforms are evolving beyond traditional, monolithic software by enabling the automated transformation of graphical process representations into scalable, cloud-native computational workflows. In this approach, a user can design a chemical process using a graphical user interface or a process flow diagram, where each unit operation and stream is visually arranged and parameterized. This graphical or graph-based representation is then automatically converted into a structured, machine-readable format—such as XML, JSON, or a domain-specific schema—that captures the topology, parameters, and interconnections of the process. From this structured representation, the system generates infrastructure-as-code (IaC) artifacts, such as Terraform or CloudFormation templates, which define the deployment of simulation components as modular, distributed services in a cloud environment. Each process module (e.g., reactor, separator, heat exchanger) is mapped to a corresponding compute resource, such as a serverless function, container, or virtual machine, and the data flows between modules are orchestrated using message queues or event-driven architectures. This pipeline enables rapid, reproducible, and scalable deployment of complex chemical simulations, supporting high-throughput studies, real-time optimization, and flexible integration with modern IT infrastructure.

Building on this foundation, advanced systems leverage large language models (LLMs) and neuro-symbolic AI to further automate and enhance the simulation lifecycle. LLMs are employed to interpret and reason over both the graphical and structured representations of chemical processes, enabling the extraction of process logic, validation of configuration, and translation between different modalities (e.g., GUI, XML, IaC, and executable code). These AI agents can generate or refine infrastructure templates, suggest optimal deployment strategies, and even synthesize simulation code tailored to specific process requirements. By integrating symbolic reasoning—such as enforcing physical laws, process constraints, and domain ontologies—with the generative capabilities of LLMs, the system can ensure scientific fidelity, detect configuration errors, and adapt workflows dynamically. This hybrid approach supports intelligent orchestration of distributed simulations, automated model selection, and semantic error checking, ultimately bridging the gap between domain expertise and computational execution in chemical engineering. Optional features may include the use of multi-agent architectures for task decomposition, feedback-driven optimization loops, and integration with external data sources or simulation engines.

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

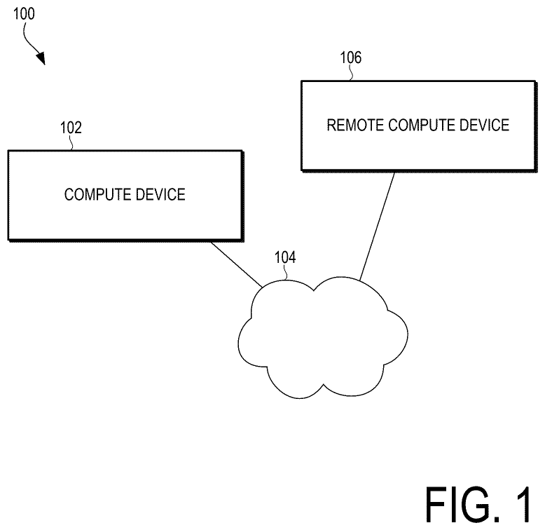

Referring now to FIG. 1, in one embodiment, a system 100 for performing chemical simulations includes one or more compute devices 102 connected by a network 104. The compute devices 102 may be embodied as, e.g., laptop computers, desktop computers, tablets, cell phones, and/or the like. The network may be embodied as a local network, the Internet, a cellular network, and/or the like. The system 100 may include two compute devices 102 communicating with each other or may include three or more compute devices 102 in a teleconference. In use, information related to a chemical simulation, such as a graphical representation, an XML representation, infrastructure-as-code (IAC) information, etc., may be sent between the compute device 102, 106, as described in more detail below.

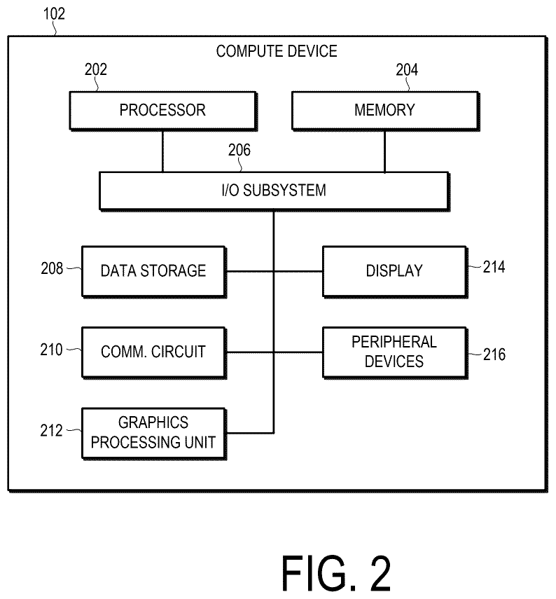

Referring now to FIG. 2, in one embodiment, a compute device 102 for performing chemical simulations is shown. The compute device 102 may be embodied as any type of compute device. For example, the compute device 102 may be embodied as or otherwise be included in, without limitation, a server computer, an embedded computing system, a System-on-a-Chip (SoC), a multiprocessor system, a processor-based system, a consumer electronic device, a smartphone, a cellular phone, a desktop computer, a tablet computer, a notebook computer, a laptop computer, a network device, a router, a switch, a networked computer, a wearable computer, a handset, a messaging device, a camera device, and/or any other compute device. In some embodiments, the compute device 102 may be located in a data center, such as an enterprise data center (e.g., a data center owned and operated by a company and typically located on company premises), managed services data center (e.g., a data center managed by a third party on behalf of a company), a colocation data center (e.g., a data center in which data center infrastructure is provided by the data center host and a company provides and manages their own data center components (servers, etc.)), cloud data center (e.g., a data center operated by a cloud services provider that host companies applications and data), and an edge data center (e.g., a data center, typically having a smaller footprint than other data center types, located close to the geographic area that it serves).

The illustrative compute device 102 includes a processor 202, a memory 204, an input/output (I/O) subsystem 206, data storage 208, a communication circuit 210, a display 212, and one or more peripheral devices 214. In some embodiments, one or more of the illustrative components of the compute device 102 may be incorporated in, or otherwise form a portion of, another component. For example, the memory 204, or portions thereof, may be incorporated in the processor 202 in some embodiments. In some embodiments, one or more of the illustrative components may be physically separated from another component.

The processor 202 may be embodied as any type of processor capable of performing the functions described herein. For example, the processor 202 may be embodied as a single or multi-core processor(s), a single or multi-socket processor, a digital signal processor, a graphics processor, a neural network compute engine, an image processor, a microcontroller, or other processor or processing/controlling circuit. Similarly, the memory 204 may be embodied as any type of volatile or non-volatile memory or data storage capable of performing the functions described herein. In operation, the memory 204 may store various data and software used during operation of the compute device 102 such as operating systems, applications, programs, libraries, and drivers. The memory 204 is communicatively coupled to the processor 202 via the I/O subsystem 206, which may be embodied as circuitry and/or components to facilitate input/output operations with the processor 202, the memory 204, and other components of the compute device 102. For example, the I/O subsystem 206 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. The I/O subsystem 206 may connect various internal and external components of the compute device 102 to each other with use of any suitable connector, interconnect, bus, protocol, etc., such as an SoC fabric, PCIe®, USB2, USB3, USB4, NVMe®, Thunderbolt®, and/or the like. In some embodiments, the I/O subsystem 206 may form a portion of a system-on-a-chip (SoC) and be incorporated, along with the processor 202, the memory 204, and other components of the compute device 102 on a single integrated circuit chip.

The data storage 208 may be embodied as any type of device or devices configured for the short-term or long-term storage of data. For example, the data storage 208 may include any one or more memory devices and circuits, memory cards, hard disk drives, solid-state drives, or other data storage devices.

The communication circuit 210 may be embodied as any type of interface capable of interfacing the compute device 102 with other compute devices, such as over one or more wired or wireless connections. In some embodiments, the communication circuit 210 may be capable of interfacing with any appropriate cable type, such as an electrical cable or an optical cable. The communication circuit 210 may be configured to use any one or more communication technology and associated protocols (e.g., Ethernet, Bluetooth®, Wi-Fi®, WiMAX, near field communication (NFC), etc.). The communication circuit 210 may be located on silicon separate from the processor 202, or the communication circuit 210 may be included in a multi-chip package with the processor 202, or even on the same die as the processor 202. The communication circuit 210 may be embodied as one or more add-in-boards, daughtercards, network interface cards, controller chips, chipsets, specialized components such as a field-programmable gate array (FPGA) or application-specific integrated circuit (ASIC), or other devices that may be used by the compute device 202 to connect with another compute device. In some embodiments, communication circuit 210 may be embodied as part of a system-on-a-chip (SoC) that includes one or more processors or included on a multichip package that also contains one or more processors. In some embodiments, the communication circuit 210 may include a local processor (not shown) and/or a local memory (not shown) that are both local to the communication circuit 210. In such embodiments, the local processor of the communication circuit 210 may be capable of performing one or more of the functions of the processor 202 described herein. Additionally or alternatively, in such embodiments, the local memory of the communication circuit 210 may be integrated into one or more components of the compute device 202 at the board level, socket level, chip level, and/or other levels.

The graphics processing unit (GPU) 212 may be embodied as any type of specialized processor designed to accelerate the rendering of images, video, and animations, as well as to perform parallel computations for scientific, engineering, or machine learning tasks. In some embodiments, the GPU 212 may be integrated directly onto the same die as the central processor 202 (as in a system-on-a-chip architecture), or it may be provided as a discrete component connected via a high-speed interface such as PCI Express (PCIe®). The GPU 212 may include its own dedicated memory, often referred to as video RAM (VRAM), which is optimized for high-bandwidth data access required for graphics and compute workloads. In operation, the GPU 212 may be responsible for processing and outputting graphical data to the display 214, offloading intensive graphical computations from the main processor 202 to improve overall system performance. Additionally, in some embodiments, the GPU 212 may be leveraged for general-purpose computing tasks (GPGPU), such as accelerating simulations, machine learning inference, or other parallelizable workloads relevant to chemical engineering simulations. The GPU 212 may support industry-standard graphics APIs (e.g., OpenGL®, DirectX®, Vulkan®) and compute APIs (e.g., CUDA®, OpenCL™), enabling flexible integration with a wide range of software applications and simulation environments. In some embodiments, multiple GPUs may be present to further enhance computational throughput and graphical capabilities.

The display 214 may be embodied as any type of display on which information may be displayed to a user of the compute device 102, such as a touchscreen display, a liquid crystal display (LCD), a thin film transistor LCD (TFT-LCD), a light-emitting diode (LED) display, an organic light-emitting diode (OLED) display, a cathode ray tube (CRT) display, a plasma display, an image projector (e.g., 2D or 3D), a laser projector, a heads-up display, and/or other display technology. The display 214 may have any suitable resolution, such as 7680×4320, 3840×2160, 1920×1200, 1920×1080, etc.

In some embodiments, the compute device 102 may include other or additional components, such as those commonly found in a compute device. For example, the compute device 102 may also have peripheral devices 216, such as a keyboard, a mouse, a speaker, an external storage device, a battery, etc. In some embodiments, the compute device 102 may be connected to a dock that can interface with various devices, including peripheral devices 216.

The compute device 104 may include similar or the same components as the compute device 102, which will not be repeated in the interest of clarity.

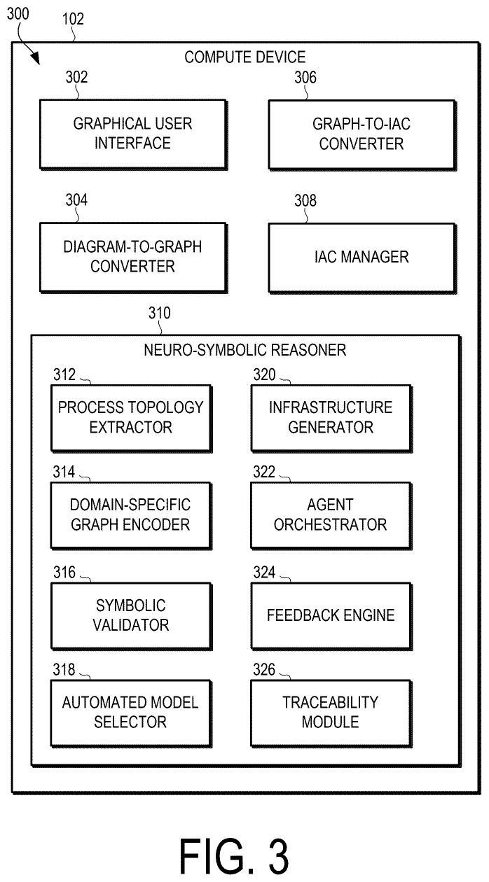

Referring now to FIG. 3, in an illustrative embodiment, the compute device 102 establishes an environment 300 during operation. Although the environment is described in regard to the compute device 102, it should be appreciated that any suitable component of the environment 300 may be embodied within any suitable combination of one or more compute devices 102 and/or one or more remote compute devices 104. The illustrative environment 300 includes a graphical user interface (GUI) 302, diagram-to-graph converter 304, graph-to-IAC converter 306, IAC manager 308, and neuro-symbolic reasoner 310. The various modules of the environment 300 may be embodied as hardware, software, firmware, or a combination thereof. For example, the various modules, logic, and other components of the environment 300 may form a portion of, or otherwise be established by, the processor 202, the memory 204, the data storage 208, or other hardware components of the compute device 102. As such, in some embodiments, one or more of the modules of the environment 300 may be embodied as circuitry or collection of electrical devices (e.g., GUI circuitry 302, diagram-to-graph converter circuitry 304, graph-to-IAC converter circuitry 306, etc.). It should be appreciated that, in such embodiments, one or more of the circuits (e.g., the GUI circuitry 302, the diagram-to-graph converter circuitry 304, the graph-to-IAC converter circuitry 306, etc.) may form a portion of one or more of the processor 202, the memory 204, the I/O subsystem 206, the data storage 208, the GPU 212, and/or other components of the compute device 102. For example, in some embodiments, some or all of the modules may be embodied as the processor 202 and/or GPU 212 as well as the memory 204 and/or data storage 208 storing instructions to be executed by the processor 202 and/or GPU 212. Additionally, in some embodiments, one or more of the illustrative modules may form a portion of another module and/or one or more of the illustrative modules may be independent of one another. Further, in some embodiments, one or more of the modules of the environment 300 may be embodied as virtualized hardware components or emulated architecture, which may be established and maintained by the processor 202 or other components of the compute device 102. It should be appreciated that some of the functionality of one or more of the modules of the environment 300 may require a hardware implementation, in which case embodiments of modules that implement such functionality will be embodied at least partially as hardware.

The GUI 302, which may be embodied as hardware, firmware, software, virtualized hardware, emulated architecture, and/or a combination thereof, as discussed above, is configured to be a comprehensive graphical user interface module designed to facilitate the intuitive creation, configuration, and management of chemical engineering simulations. It serves as the primary point of interaction between the user and the underlying simulation system, enabling engineers, scientists, and process designers to visually construct and manipulate process flow diagrams that represent complex chemical processes. Through the GUI, users can select from a palette of chemical process modules—such as reactors, compressors, heat exchangers, separators, and other unit operations—and place them onto a virtual workspace. Each module can be connected to others by drawing streams or channels, which represent the flow of materials, energy, or information between process units. This visual approach allows users to build detailed process schemes without the need to write code or manually edit configuration files.

The GUI 302 may be implemented as a software application, which may be delivered as a desktop program, a web-based interface, or as a component of a larger cloud-based simulation platform. In some embodiments, the GUI may also be realized in hardware or as a combination of hardware and software, such as on a touchscreen-enabled device or a specialized workstation. Regardless of the implementation, the GUI is designed to be highly interactive and user-friendly, supporting drag-and-drop operations, context-sensitive menus, and real-time feedback as the user constructs or modifies the process diagram. Users can click on individual modules to access detailed configuration panels, where they can specify parameters such as operating conditions, material properties, and model selection. The GUI may also provide validation features, highlighting incomplete connections or missing parameters to help ensure that the process diagram is logically consistent and ready for simulation.

Beyond process construction, the GUI 302 supports the import and export of process diagrams in various structured formats, such as XML or JSON, enabling interoperability with other engineering tools and facilitating the transition from graphical design to automated simulation workflows, as described in more detail below. Users can save their work, load existing process schemes, and generate reports or documentation directly from the interface. The GUI may also include features for zooming, panning, and organizing large or complex diagrams, as well as search and filtering tools to quickly locate specific modules or streams within a project.

The primary purpose of the GUI 302 is to lower the barrier to entry for chemical process simulation, making it accessible to users who may not have expertise in programming or infrastructure configuration. By providing a visual, interactive environment, the GUI streamlines the process of designing, configuring, and managing simulations, allowing users to focus on engineering decisions rather than technical implementation details. The GUI is tightly integrated with the rest of the simulation system, ensuring that the graphical representation of the process is faithfully translated into the structured data and computational artifacts required for automated, scalable simulation execution. Through its design, the GUI 302 enables rapid prototyping, iterative design, and efficient communication of process concepts among team members, ultimately supporting more agile and effective chemical engineering workflows.

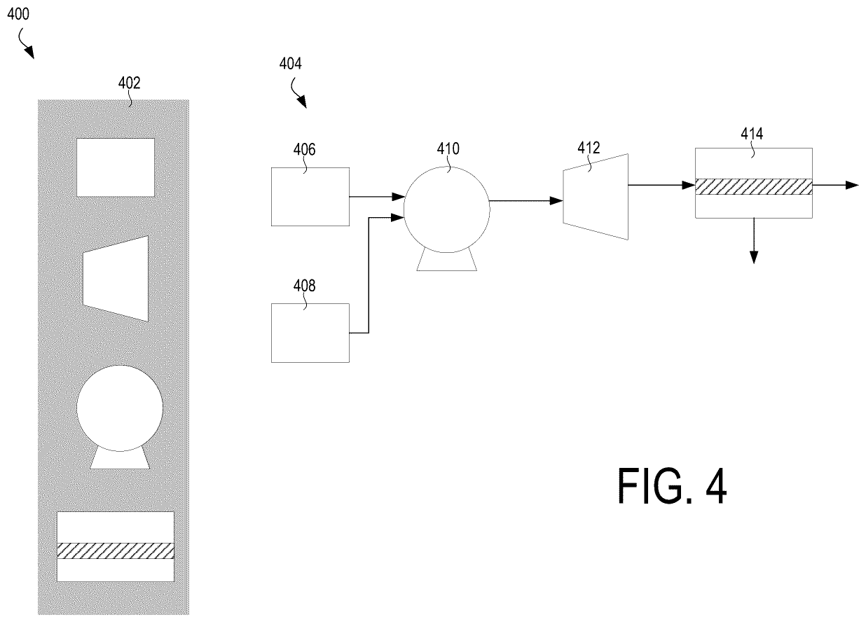

FIG. 4 illustrates an example embodiment of the graphical user interface (GUI) 400 for a chemical engineering simulation system. The GUI is organized into two main regions: a selection area 402 and a process flow workspace 404. The selection area 402 is a palette of available process modules, each represented by a distinct icon corresponding to a chemical engineering component. Users interact with the GUI by dragging components from the selection area 402 into the workspace 404, where they can arrange, connect, and configure them to model a desired chemical process.

In the depicted example, the process flow begins with a reagent source 406, which may represent a feed tank, a supply vessel, or an input stream containing raw materials or reactants. The reagent source 406 is connected to a mixer 410, which serves to homogenize the composition, temperature, or other properties of the process fluid. The mixer 410 may receive additional input from a secondary reagent source 408, allowing for the blending of multiple streams or the introduction of additives, catalysts, or secondary reactants. compressor 408, which increases the pressure of the incoming stream. The output of the mixture 412 then enters a compressor 412, which increases the pressure of the incoming stream. This step is present in many chemical processes, as elevated pressure can enhance reaction rates, improve separation efficiency, or enable downstream unit operations to function within their design parameters.

Following compressing, the process stream is directed into a membrane unit 414. The membrane 414 is a separation device that exploits differences in permeability, molecular size, or chemical affinity to selectively partition components of the mixture. For example, in a gas separation process, the membrane may preferentially allow the passage of certain gases (such as hydrogen or carbon dioxide) while retaining others. The membrane unit 414 typically produces at least two output streams: a permeate (the fraction that passes through the membrane) and a retentate (the fraction that is retained). These outputs can be further processed, recycled, or collected as final products.

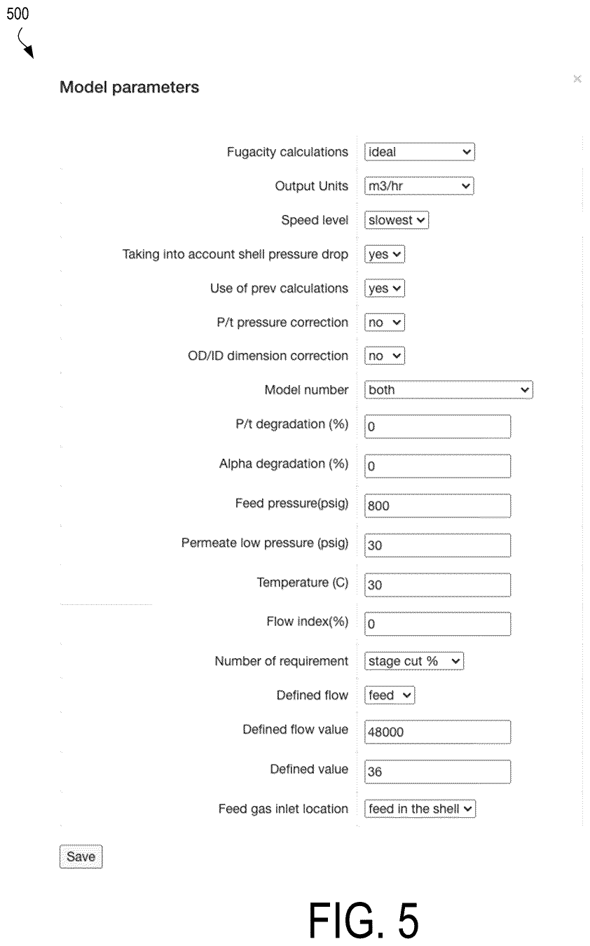

Referring now to FIG. 5, an illustrative embodiment of a graphical user interface (GUI) element 500 is shown, which is designed to facilitate the configuration and management of model parameters within a chemical engineering simulation environment. The GUI element 500 presents a comprehensive panel that enables users to specify, adjust, and review a wide range of technical parameters associated with a particular process module, such as a membrane unit, reactor, or other unit operation.

In the depicted embodiment, the GUI element 500 includes a series of input fields, dropdown menus, and selection boxes, each corresponding to a distinct model parameter. These parameters encompass both physical and operational characteristics relevant to the simulation of chemical processes. For example, the user may select the type of fugacity calculations to be employed (e.g., ideal or real), specify the output units (such as m³/hr), and choose the desired speed level for the simulation. Additional options allow the user to indicate whether shell pressure drop should be taken into account, whether previous calculations should be used, and whether corrections for pressure and temperature (P/t) or for outer diameter/inner diameter (OD/ID) dimensions should be applied.

The GUI element 500 further provides fields for entering numerical values for key process variables, including feed pressure (psig), permeate low pressure (psig), temperature (°C), and flow index (%). Users may also define model-specific attributes such as model number, degradation percentages for pressure/temperature and alpha parameters, and the number of requirements (e.g., stage cut percentage). The interface supports the specification of defined flow types and values, as well as the location of the feed gas inlet (for example, whether the feed is introduced into the shell).

Each parameter is presented in a clear, organized manner, with appropriate input controls to ensure accurate and efficient data entry. The interface may include validation logic to check for completeness and consistency of the entered values, and may provide contextual help or tooltips to assist users in understanding the significance of each parameter.

Upon completion of the parameter configuration, the user may activate a “Save” button to store the specified settings, which are then incorporated into the simulation workflow. The GUI element 500 is designed to support rapid prototyping, iterative design, and detailed customization of process modules, enabling engineers and process designers to tailor simulation models to the specific requirements of their chemical processes.

In some embodiments, the GUI element 500 may be integrated with other components of the simulation environment, such as real-time feedback displays, validation engines, or automated model selection modules. This integration allows for dynamic updating of parameters, immediate visualization of simulation results, and seamless transition between design and execution phases. The flexible and extensible nature of the GUI element 500 supports a wide range of chemical engineering applications, from laboratory-scale experiments to large-scale industrial process simulations.

The GUI enables users to define the connectivity and flow direction between components by drawing channels or streams, which are visually represented as arrows or lines. Each connection can be configured with parameters such as flow rate, pressure, temperature, and composition, allowing for detailed simulation of process behavior. The user can click on any component to access a configuration panel, where device-specific parameters (e.g., compressor pressure ratio, mixer residence time, membrane selectivity) can be specified. The GUI may also provide real-time validation, alerting the user to incomplete connections, missing parameters, or logical inconsistencies in the process scheme.

Potential components that might be included in a chemical engineering system, and for which the GUI could provide elements, includes: feed tanks, reagent sources, product tanks, compressors, pumps, valves, heat exchangers (shell-and-tube, plate, air-cooled), reactors (continuous stirred-tank reactor (CSTR), plug flow reactor (PFR), batch reactor, catalytic reactor, fixed-bed reactor, fluidized-bed reactor), mixers, separators (distillation columns, absorption columns, extraction units, membrane units, centrifuges, decanters), filters (microfiltration, ultrafiltration, nanofiltration, reverse osmosis), crystallizers, evaporators, condensers, scrubbers, dryers, storage vessels, recycle loops, sampling ports, sensors (temperature, pressure, flow, composition), control elements (PID controllers, actuators), and utility connections (steam, cooling water, inert gas).

The interaction between these components is governed by the principles of chemical engineering and process systems engineering. For example, a feed tank may supply reactants to a pump, which delivers the stream at a controlled flow rate to a reactor. The reactor transforms the chemical composition via reaction kinetics, producing an effluent that is sent to a heat exchanger for temperature adjustment. The cooled or heated stream may then enter a separator, such as a distillation column, where components are partitioned based on volatility. The separated fractions can be routed to product tanks, recycled to upstream units, or sent to waste treatment. Control elements and sensors are distributed throughout the system to monitor process variables and maintain safe, efficient operation. Recycle loops allow for the reuse of unreacted materials, while utility connections provide necessary energy or inert environments.

In the GUI, each of these components is represented by a distinct icon or graphical element, and their interactions are modeled by connecting streams. The user can simulate complex process networks, including parallel and series arrangements, feedback loops, and multi-stage operations. The GUI supports the configuration of process parameters, the visualization of process flows, and the export of the entire scheme to structured formats for further simulation, optimization, or deployment. This level of detail and flexibility enables the simulation of a wide range of chemical engineering processes, from simple laboratory-scale experiments to large-scale industrial plants, supporting both educational and professional applications.

The diagram-to-graph converter 304, which may be embodied as hardware, firmware, software, virtualized hardware, emulated architecture, and/or a combination thereof, as discussed above, is configured to transform the visual, user-constructed process diagrams from the graphical user interface (GUI) 302 into a structured, machine-readable graph representation. This converter is designed to operate seamlessly with the GUI 302, whether as an integrated component or as a service accessible via an application programming interface (API).

The primary function of the diagram-to-graph converter 304 is to interpret the process flow diagram as constructed by the user within the GUI workspace. In certain embodiments, the user-constructed process diagram is converted into a structured, machine-readable format, which may include XML in particular cases. However, other formats such as JSON or domain-specific schemas may also be employed. The GUI allows users to select, arrange, and connect various chemical process modules—such as reactors, compressors, mixers, membranes, and other unit operations—using intuitive drag-and-drop and connection tools. Each module is represented by a graphical icon or element, and connections between modules are depicted as lines or arrows indicating the flow of materials, energy, or information. The converter 304 systematically traverses the visual diagram, identifying each module instance, its type, its unique identifier, and its associated parameters, as well as the connectivity and directionality of each stream or channel linking the modules.

The extraction process may occur in any suitable manner. For example, in one approach, the converter 304 directly interrogates the GUI's visual elements, parsing the rendered diagram to reconstruct the underlying process topology. This may involve reading the positions, types, and properties of graphical objects, as well as the endpoints and attributes of connection lines. In another approach, the converter 304 accesses the underlying data model or state that the GUI uses to generate the visual representation. Modern GUIs typically maintain an internal data structure—such as a tree, graph, or object model—that encodes all modules, their parameters, and their interconnections. By interfacing with this data model, the converter can obtain a precise and unambiguous description of the process, independent of the visual layout or user interface artifacts.

Once the relevant information is extracted, the diagram-to-graph converter 304 encodes the process as a graph-based representation. This representation may take the form of an XML document, a JSON object, or another domain-specific schema. In this graph, each node corresponds to a process module, annotated with metadata such as module type (e.g., CSTR, membrane, compressor), unique identifier, user-specified parameters (e.g., volume, pressure, temperature, selectivity), and any default or inferred attributes. Edges in the graph represent the streams or channels connecting modules, and are annotated with properties such as flow direction, stream type (material, energy, information), and stream-specific parameters (e.g., flow rate, composition, pressure, temperature). The graph may also encode higher-level constructs, such as recycle loops, parallel or series arrangements, and hierarchical groupings of modules.

The converter 304 is capable of capturing additional technical information from the GUI or its data model, including module configuration states, validation flags (e.g., whether a connection is complete or a parameter is missing), and user annotations or comments. It may also extract information about the spatial arrangement of modules, which can be useful for layout preservation or for mapping to physical plant topologies. The resulting graph representation is structured to be both human-readable and machine-interpretable, supporting downstream processing such as simulation code generation, infrastructure-as-code synthesis, or integration with external engineering tools.

In advanced embodiments, the diagram-to-graph converter 304 may incorporate semantic analysis to infer implicit relationships or to validate the logical consistency of the process diagram. For example, it can check for unconnected modules, circular dependencies, or parameter mismatches, and encode these findings in the graph metadata. The converter may also support extensibility, allowing new module types or connection rules to be defined and incorporated without requiring changes to the core logic.

Overall, the diagram-to-graph converter 304 serves as a bridge between the user's intuitive, graphical process design and the formal, structured representations required for automated, scalable chemical engineering simulation. By faithfully capturing all relevant technical details from the GUI or its underlying data, the converter ensures that the process logic, parameters, and topology are preserved and made available for subsequent computational workflows.

The graph-to-IAC converter 306, which may be embodied as hardware, firmware, software, virtualized hardware, emulated architecture, and/or a combination thereof, as discussed above, is configured to transform the structured, machine-readable graph representation of a chemical process (such as one produced by the diagram-to-graph converter 304) into infrastructure-as-code (IAC) artifacts or other executable deployment formats. This module may be realized as a standalone service, a library, a microservice within a distributed architecture, or even as a dedicated hardware accelerator for high-throughput environments.

The graph-to-IAC converter 306 ingests a graph-based representation of a chemical process, where nodes correspond to process modules (such as reactors, compressors, heat exchangers, separators, and so forth) and edges represent the interconnections or streams (material, energy, or information flows) between these modules. Each node and edge is annotated with metadata, including module types, unique identifiers, user-specified or default parameters, and stream properties such as flow direction, composition, and operating conditions. The converter parses this graph, extracting the full topology, parameterization, and connectivity of the process.

In an example embodiment, the converter traverses the graph to identify all modules and their relationships, then maps each module to a corresponding computational or infrastructure resource. For example, a reactor node may be mapped to a serverless function (such as an AWS Lambda function), a containerized microservice (e.g., Docker container orchestrated by Kubernetes or AWS ECS), or a virtual machine instance (such as an EC2 instance). The mapping logic can be rule-based, data-driven, or AI-assisted, allowing for flexible deployment strategies based on module complexity, resource requirements, or user preferences. The converter generates IAC artifacts—such as Terraform, AWS CloudFormation, Pulumi, or Ansible templates—that define the provisioning, configuration, and orchestration of these resources in a cloud or hybrid environment. Each artifact specifies the resource type, configuration parameters (e.g., memory, CPU, environment variables), networking details, and dependencies, ensuring that the deployed infrastructure mirrors the logical process graph.

The converter 306 encodes the data flows between modules as message queues, event triggers, or direct network connections, depending on the target platform. For instance, material streams between modules may be represented as messages in systems such as AWS SQS queues, Kafka topics, or RESTful API endpoints, with the IAC template specifying the creation and wiring of these communication channels. The converter can generate additional glue code or configuration files to handle serialization, deserialization, and validation of messages, ensuring robust and type-safe data exchange between process modules.

The converter 306 encodes the data flows between modules as message queues, event triggers, or direct network connections, depending on the target platform. In an illustrative embodiment, that these streams are implemented as messages in systems such as AWS Simple Queue Service (SQS). For example, material streams between modules may be represented by SQS queues, Kafka topics, or RESTful API endpoints, with the infrastructure-as-code (IAC) template specifying the creation and configuration of these communication channels. The converter may also generate supporting code or configuration files to manage the serialization, deserialization, and validation of messages, thereby ensuring robust and type-safe data exchange between process modules.

In more advanced embodiments, the graph-to-IAC converter supports multiple output modalities beyond traditional IAC. It may generate workflow definitions for serverless orchestration engines, such as AWS Step Functions, Azure Durable Functions, or Google Cloud Workflows, encoding the process graph as a directed acyclic graph (DAG) of tasks with explicit execution order, branching, and error handling logic. The converter can also produce domain-specific simulation scripts, such as Python or MATLAB code, that instantiate and connect process modules according to the graph topology, enabling local or hybrid execution outside of a cloud environment. In some cases, the converter may target industrial automation platforms, generating PLC ladder logic, OPC UA configurations, or SCADA system scripts that mirror the process structure for deployment in physical plants.

The module is designed to be highly extensible and configurable. It may include a plugin architecture that allows new resource types, deployment targets, or code generators to be added without modifying the core logic. The mapping from graph elements to infrastructure resources can be customized via user-defined templates, AI-driven optimization, or policy-based rules that account for cost, performance, security, or compliance requirements. The converter may also support round-trip engineering, where changes in the deployed infrastructure can be reflected back into the graph representation, enabling bidirectional synchronization and iterative design.

To ensure correctness and reliability, the graph-to-IAC converter performs extensive validation and error checking. It verifies that all required parameters are specified, that module interconnections are logically consistent, and that resource dependencies are properly resolved. The converter may simulate the deployment plan, providing users with a preview of the resulting infrastructure and highlighting potential issues such as circular dependencies, resource conflicts, or unsupported configurations. In some embodiments, the converter integrates with version control systems, enabling collaborative editing, change tracking, and rollback of IAC artifacts.

In summary, the graph-to-IAC converter 306 serves as a bridge between high-level, graphical process design and automated, scalable deployment of chemical engineering simulations. By translating structured process graphs into IAC templates, workflow definitions, simulation scripts, or industrial automation code, it enables rapid, reproducible, and flexible execution of complex chemical processes across a wide range of computational and physical environments. Its modular, extensible architecture supports a variety of deployment strategies, output formats, and integration scenarios, making it a foundational component for modern, cloud-native chemical engineering platforms.

The IAC manager 308, which may be embodied as hardware, firmware, software, virtualized hardware, emulated architecture, and/or a combination thereof, as discussed above, is configured to orchestrate the deployment, execution, and management of infrastructure-as-code (IAC) artifacts generated by the graph-to-IAC converter 306. This module is designed to operate seamlessly within the broader simulation platform, interfacing with both the underlying computational infrastructure and the user-facing graphical user interface (GUI).

Upon receiving IAC artifacts—such as Terraform, CloudFormation, or other declarative templates—from the graph-to-IAC converter, the IAC manager 308 parses and validates these artifacts to ensure syntactic correctness, resolve dependencies, and verify that all required parameters and resources are specified. The manager then initiates the provisioning of computational resources as defined in the IAC, which may include serverless functions, containers, virtual machines, message queues, storage buckets, and networking components. This provisioning process is tightly coupled with the scheduling logic of the IAC manager 308, which determines the optimal order and timing for resource creation, taking into account dependencies between simulation modules, resource availability, and user-specified constraints such as cost, performance, or security requirements.

Once the infrastructure is provisioned, the IAC manager 308 is responsible for scheduling and executing simulation tasks across the distributed resources. It interprets the logical execution graph, derived from the original process topology, and maps each simulation module (e.g., reactor, separator, heat exchanger) to its corresponding compute resource. The manager coordinates the flow of data between modules, leveraging message queues, event-driven triggers, or direct network connections as specified in the IAC. It monitors the status of each task, handling retries, error detection, and recovery in the event of failures or resource contention. The IAC manager may also implement dynamic scaling policies, automatically adjusting the number of compute instances or parallel executions based on workload, simulation complexity, or real-time feedback from the running processes.

As simulation tasks complete, the IAC manager 308 collects and aggregates output data from the various compute resources. This may involve retrieving logs, intermediate results, or final simulation outputs from distributed storage locations, message queues, or direct API responses. The manager performs post-processing as needed, such as data validation, aggregation, statistical analysis, or transformation into user-friendly formats. It maintains a comprehensive record of the simulation execution, including resource utilization, execution timelines, and any encountered errors or warnings, supporting both auditability and reproducibility.

In some embodiments, the IAC manager 308 presents simulation results to the user. The IAC manager 308 interfaces with the GUI 302, delivering output data, visualizations, and status updates in real time or upon completion of the simulation. Users can view detailed reports, graphical representations of process flows, performance metrics, and diagnostic information directly within the GUI. The IAC manager 308 supports interactive features, allowing users to drill down into specific modules, inspect parameter values, and review execution logs. The manager enables a feedback or closed-loop revision process: users can analyze the results, adjust process parameters or configurations within the GUI, and trigger iterative re-deployment and re-execution of the simulation. This closed-loop capability fosters rapid prototyping, optimization, and continuous improvement of chemical process designs, ensuring that the simulation environment remains agile, responsive, and aligned with user objectives. Through its comprehensive management of IAC execution, resource orchestration, data handling, and user interaction, the IAC manager 308 serves as the operational backbone of the cloud-native chemical engineering simulation platform.

The neuro-symbolic reasoner 310, which may be embodied as hardware, firmware, software, virtualized hardware, emulated architecture, and/or a combination thereof, as discussed above, serves as an intelligent, hybrid module that bridges advanced artificial intelligence with the rigorous demands of chemical engineering simulation. This component is designed to integrate the generative and interpretive capabilities of large language models (LLMs) and neuro-symbolic neural networks with the structured, rule-based logic of symbolic reasoning, enabling a new level of automation, adaptability, and scientific fidelity in process simulation workflows. At its core, the neuro-symbolic reasoner 310 interprets and transforms high-level process representations—whether originating from graphical user interfaces, structured files, or natural language descriptions—into actionable, machine-readable formats that can be validated, optimized, and executed within a distributed, cloud-native environment.

The neuro-symbolic reasoner 310, which may be embodied as hardware, firmware, software, virtualized hardware, emulated architecture, and/or a combination thereof, serves as an intelligent, hybrid module that bridges advanced artificial intelligence with the rigorous demands of chemical engineering simulation. In certain embodiments, the neuro-symbolic reasoner 310 includes both large language model (LLM) components and logic programming components, which may operate in parallel and in sequence to interpret, validate, and optimize process representations. The LLM component provides generative and interpretive capabilities, while the logic programming component implements structured, rule-based reasoning to verify and improve the outputs produced by the LLM. This architecture enables the neuro-symbolic reasoner 310 to transform high-level process representations—whether originating from graphical user interfaces, structured files, or natural language descriptions—into actionable, machine-readable formats that can be validated, optimized, and executed within a distributed, cloud-native environment. In some embodiments, the logic programming components are created or guided by the LLM, and act to ensure scientific fidelity and correctness in process simulation workflows.

The reasoner's functions span a broad spectrum of tasks that collectively enable end-to-end automation and intelligent orchestration of chemical simulations. It is capable of parsing and understanding graphical or symbolic process diagrams, extracting the underlying process topology, and encoding this information as structured graphs that capture both the connectivity and the operational parameters of each module. Leveraging the strengths of LLMs, the reasoner 310 can translate between different modalities—such as converting a process flow diagram into an XML schema, or generating infrastructure-as-code artifacts that map process modules to cloud resources. Beyond translation, the neuro-symbolic reasoner 310 applies domain-specific rules and ontologies separate from the LLM to validate process configurations, ensuring logical consistency, completeness, and adherence to physical laws. It can detect and explain errors, suggest corrections, and enforce constraints that are important for reliable simulation.

Another function of the neuro-symbolic reasoner 310 is its ability to automate model selection and configuration, dynamically choosing the most appropriate mathematical or computational models for each process module based on context, user goals, or available data. The reasoner 310 also supports feedback-driven optimization, analyzing simulation results and iteratively refining process parameters or configurations to achieve desired outcomes. Its architecture is inherently modular and extensible, supporting multi-agent collaboration where specialized sub-modules handle tasks such as code generation, workflow orchestration, and semantic error checking. By combining neural and symbolic approaches, the neuro-symbolic reasoner 310 ensures that the entire simulation pipeline is not only automated and scalable, but also explainable, auditable, and aligned with the rigorous standards of chemical engineering practice.

The present disclosure introduces a hybrid neuro-symbolic architecture that fundamentally transforms how chemical engineering simulations are designed, executed, and managed in distributed environments. At its core, this system fuses the generative and interpretive capabilities of large language models (LLMs) with the rigor and transparency of symbolic reasoning, creating a platform that is both highly automated and scientifically robust. Unlike conventional simulation tools that rely on static, monolithic models and proprietary interfaces, the neuro-symbolic approach enables dynamic, multi-modal interaction with chemical process representations—whether they originate from graphical user interfaces, structured XML files, or infrastructure-as-code (IaC) templates.

In this architecture, LLMs serve as intelligent agents that can parse and understand complex process diagrams, extract the underlying topology and operational parameters, and translate these into machine-readable formats suitable for automated deployment. The LLMs are not limited to simple text generation; they are fine-tuned to interpret domain-specific language, recognize chemical engineering concepts, and generate executable code or configuration artifacts that reflect the intent and constraints of the process designer. For example, an LLM can convert a visual process flow diagram into a structured graph, validate its logical consistency, and produce IaC templates that map each unit operation to a corresponding cloud resource, such as a serverless function or containerized microservice.

Layered atop the neural capabilities of the LLM is a symbolic reasoning engine that enforces scientific and engineering constraints throughout the simulation lifecycle. This symbolic layer leverages domain ontologies, rule sets, and physical laws to validate the outputs of the LLM, ensuring that generated configurations adhere to principles such as mass and energy conservation, proper connectivity, and operational safety. Symbolic reasoning also provides explainability, allowing users and auditors to trace the logic behind simulation decisions, detect errors, and understand how process parameters and module interactions are determined. For instance, if the LLM proposes a process configuration that violates a thermodynamic constraint or introduces a circular dependency, the symbolic reasoner can flag the issue, suggest corrections, and document the rationale for any changes.

The hybrid neuro-symbolic system is inherently modular and extensible, supporting the integration of new process modules, simulation engines, and deployment targets without sacrificing scientific fidelity or transparency. By combining the adaptability and generalization power of neural networks with the deterministic, rule-based logic of symbolic AI, the hybrid neuro-symbolic system enables intelligent orchestration of distributed chemical simulations, automated model selection, and semantic error checking. This approach not only accelerates the design and optimization of chemical processes but also bridges the gap between domain expertise and computational execution, making advanced simulation capabilities accessible to a broader range of users while maintaining the highest standards of reliability and auditability.

In some embodiments, the neuro-symbolic reasoner 310 may be able to implement or augment the functionalities of other components in the system, such as the graphical user interface (GUI) 302, the diagram-to-graph converter 304, the graph-to-IAC converter 306, and/or the IAC manager 308. By leveraging its hybrid capabilities—combining the generative and interpretive strengths of large language models (LLMs) with the rigor and transparency of symbolic reasoning—the reasoner 310 can serve as the central orchestrator for the entire simulation workflow.

For example, the reasoner 310 can subsume the role of the GUI 302 by interpreting user interactions, graphical process diagrams, or even natural language descriptions, and translating them into structured, machine-readable representations. Through advanced language and vision models, the reasoner can extract the topology, parameters, and interconnections of process modules directly from the user's graphical input or textual instructions, effectively bridging the gap between human intent and computational representation. This enables a more flexible and adaptive user experience, where the system can understand and validate process designs regardless of the input modality.

Similarly, the reasoner 310 can perform the functions of the diagram-to-graph converter 304 by parsing visual or structured process diagrams and encoding them as formal graph representations. Its neuro-symbolic architecture allows it to recognize domain-specific patterns, infer implicit relationships, and validate the logical consistency of the process topology. The reasoner can also enhance this conversion by applying domain ontologies and rule-based checks, ensuring that the resulting graph accurately reflects both the user's design and the underlying physical constraints of chemical engineering.

In the context of the graph-to-IAC converter 306, the reasoner 310 can automate the translation of structured process graphs into infrastructure-as-code (IAC) artifacts. By reasoning over the graph's nodes and edges, the reasoner can generate deployment templates, workflow definitions, and configuration files tailored to the target computational environment. It can optimize resource allocation, enforce dependency constraints, and adapt the IAC output based on user preferences or system requirements. This not only streamlines the deployment process but also ensures that the infrastructure faithfully implements the intended process logic.

Finally, the reasoner 310 can take on the responsibilities of the IAC manager 308 by orchestrating the execution, monitoring, and management of distributed simulation tasks. It can schedule and coordinate the flow of data between process modules, handle error detection and recovery, and provide real-time feedback to users through the GUI. By integrating symbolic reasoning with real-time data analysis, the reasoner can dynamically adjust resource allocation, optimize execution strategies, and support iterative design and optimization cycles.

A further innovative capability of the neuro-symbolic reasoner 310 is its support for inverse process synthesis. In contrast to the conventional, forward simulation workflow—where a user specifies a process flow diagram and the system computes the resulting outputs—the inverse workflow enables the system to start from user-defined process goals or desired output specifications and automatically generate a process configuration (including module selection, interconnections, and parameterization) that is expected to achieve those goals.

From an AI, LLM, or neuro-symbolic perspective, this inverse functionality leverages the generative and interpretive strengths of large language models (LLMs) and symbolic reasoning to bridge the gap between high-level objectives and executable process designs. The neuro-symbolic reasoner 310 is capable of interpreting a wide range of user inputs, including target product specifications, performance metrics (such as yield, purity, or energy efficiency), or other operational objectives. These goals may be provided through a graphical user interface, structured files (such as XML or JSON), or natural language descriptions.

Upon receiving such goals, the neuro-symbolic reasoner 310 employs advanced AI techniques to synthesize one or more candidate process diagrams that could achieve the specified outputs within any given constraints. The LLM component draws on domain knowledge, learned patterns, and historical data to propose process topologies, select appropriate unit operations (e.g., reactors, separators, heat exchangers), and suggest plausible interconnections and flow paths. The system may generate multiple alternative configurations, each representing a different approach to meeting the process objectives.

Once candidate process diagrams are generated, the neuro-symbolic reasoner 310 configures the individual process modules and their parameters, using both neural inference and symbolic rules. This includes determining suitable operating conditions, sizing, and control strategies for each module, based on the process requirements, available data, and optimization criteria. The symbolic reasoning layer ensures that the generated configurations are logically consistent, scientifically valid, and compliant with domain-specific rules, physical laws, and safety constraints.

The system then simulates the predicted performance of the candidate process configurations, either locally or in a distributed cloud environment. The neuro-symbolic reasoner 310 compares the simulation results to the original process goals and constraints, analyzing the degree to which the outputs meet the specified objectives. If the process fails to meet the goals, the system enters an iterative refinement loop, proposing adjustments to the process topology, module selection, or parameter values, guided by optimization algorithms and domain knowledge. Each iteration involves re-validation and re-simulation, with the system learning from previous results to converge toward an optimal or satisfactory process configuration. This closed-loop, feedback-driven approach enables rapid prototyping, continuous improvement, and adaptive optimization.

This inverse synthesis capability is not limited to AI-augmented implementations. The system architecture is designed to support both AI-driven and conventional, non-AI approaches. In a non-AI implementation, the inverse workflow may be realized using rule-based engines, template libraries, or manual configuration tools. For example, a set of predefined process synthesis rules or templates can be applied to map user-specified goals to process configurations, with parameter selection and validation performed using deterministic algorithms, lookup tables, or expert-defined heuristics. While such approaches may lack the flexibility, adaptability, and learning capacity of AI-based systems, they can still provide automated or semi-automated inverse process design within the same overall framework.

By supporting both AI/neuro-symbolic and non-AI modes, the neuro-symbolic reasoner 310 ensures that the inverse process synthesis functionality is broadly accessible and robust, accommodating a wide range of user needs, technical environments, and regulatory requirements. This dual-mode capability allows users to benefit from advanced automation and optimization when AI resources are available, while retaining the option for transparent, rule-based, or expert-guided process design when preferred or required.

A more detailed description of some of the internal mechanisms and advanced capabilities of the neuro-symbolic reasoner 310 will follow below, illustrating how it enables intelligent, adaptive, and scientifically robust orchestration of chemical engineering simulations across the entire system.

The neuro-symbolic reasoner 310 includes a set of specialized submodules, each designed to address a distinct aspect of chemical process simulation and orchestration. The process topology extractor 312 interprets graphical or structured process representations, identifying the connectivity and relationships among process modules. The domain-specific graph encoder 314 transforms these topologies into formal graph structures, embedding chemical engineering semantics and metadata. The symbolic validator 316 applies domain rules and physical laws to ensure logical consistency and completeness of the process configuration. The automated model selector 318 dynamically chooses the most appropriate mathematical or computational models for each module based on context and available data. The infrastructure generator 320 translates validated process graphs into infrastructure-as-code artifacts, workflow definitions, or simulation scripts tailored to the target computational environment. The agent orchestrator 322 coordinates the execution of distributed simulation tasks, managing data flow, scheduling, and error recovery across cloud resources. The feedback engine 324 analyzes simulation results, supports iterative optimization, and enables closed-loop refinement of process parameters. Finally, the traceability module 326 maintains comprehensive records of simulation decisions, configurations, and execution outcomes, ensuring auditability and scientific transparency throughout the workflow.

The process topology extractor 312 is a specialized submodule within the neuro-symbolic reasoner 310, designed to bridge the gap between user-facing graphical representations and the formal, machine-readable process models required for automated chemical engineering simulation. Unlike conventional diagram-to-graph converters, which rely on explicit, well-structured input from graphical user interfaces (GUIs), the process topology extractor 312 leverages advanced large language models (LLMs) and neuro-symbolic AI to interpret, reason over, and reconstruct the underlying process topology from a wide range of input modalities. This includes not only standard GUI diagrams, but also incomplete, ambiguous, or multi-modal representations such as annotated images, natural language descriptions, and hybrid files combining graphical and textual elements.

The process topology extractor 312 operates by parsing the visual and symbolic elements presented in the GUI workspace, identifying each unit operation—such as reactors, compressors, heat exchangers, separators, and mixers—and mapping their interconnections, flow directions, and operational parameters. The LLM component is fine-tuned to recognize domain-specific icons, labels, and graphical conventions, enabling it to accurately distinguish between different types of chemical process modules and the streams or channels that connect them. This capability extends to interpreting user annotations, contextual cues, and even hand-drawn or non-standard diagram elements, which are often present in real-world engineering workflows.

A key innovation of the process topology extractor 312 lies in its ability to handle ambiguity and incompleteness in the input data. Traditional converters may fail or produce erroneous results when faced with missing connections, undefined parameters, or unconventional diagram layouts. In contrast, the AI-driven extractor employs both neural inference and symbolic reasoning to infer implicit relationships, validate logical consistency, and suggest plausible completions for incomplete process schemes. For example, if a user omits a connection between a feed tank and a reactor, the extractor can draw on chemical engineering principles and learned patterns to propose the most likely topology, flagging the omission for user review or automatically filling in the gap. Similarly, when process modules are labeled generically or ambiguously, the LLM can use contextual clues and domain knowledge to disambiguate their roles and assign appropriate metadata.

The process topology extractor 312 may be integrated with other modules in the environment 300, such as the diagram-to-graph converter 304 and the domain-specific graph encoder 314. After reconstructing the process topology, the extractor outputs a structured graph representation—typically in XML, JSON, or a domain-specific schema—that encodes each module as a node, annotated with type, parameters, and unique identifiers, and each stream or channel as an edge, annotated with flow properties and connectivity. This graph is then passed to the graph encoder 314 for further semantic enrichment, embedding chemical engineering ontologies and metadata that support downstream tasks such as simulation code generation, infrastructure mapping, and error checking.

In addition, the process topology extractor 312 supports multi-modal input and cross-modal translation, enabling users to interact with the simulation system through a variety of interfaces. Whether the process is described via a drag-and-drop GUI, a textual process description, or a hybrid annotated diagram, the extractor can interpret and unify these inputs into a coherent process topology. This flexibility is particularly valuable in collaborative engineering environments, where different stakeholders may contribute information in different formats, and where rapid prototyping and iterative design are important.

By combining the generative and interpretive strengths of LLMs with the rigor of symbolic reasoning, the process topology extractor 312 ensures that the reconstructed process models are both scientifically faithful and computationally robust. It enables intelligent error detection, semantic validation, and dynamic adaptation to evolving user input, supporting a seamless transition from intuitive graphical design to automated, scalable chemical simulation. This capability not only enhances the usability and accessibility of the simulation platform, but also lays the foundation for advanced features such as automated model selection, feedback-driven optimization, and integration with external data sources or simulation engines. Through its innovative approach to process topology extraction, this submodule represents a significant advancement in the automation and intelligence of chemical engineering workflows.

The domain-specific graph encoder 314 is designed to transform extracted process topologies into rich, chemically meaningful graph representations that serve as the backbone for intelligent simulation, reasoning, and automation. After the process topology extractor 312 has parsed and reconstructed the connectivity, flow directions, and operational parameters of a chemical process—whether from a graphical user interface, XML file, or natural language description—the graph encoder 314 takes this initial topology and embeds it with deep domain semantics, constraints, and expert knowledge unique to chemical engineering.

Unlike generic graph encoders, which may simply represent nodes and edges with basic attributes, the domain-specific graph encoder 314 is tailored to the nuances of chemical process systems. Each node in the graph is annotated not only with its type (such as reactor, separator, compressor, or membrane) and unique identifier, but also with a comprehensive set of metadata: physical parameters (volume, pressure, temperature, selectivity), operational constraints (maximum throughput, safety limits), and contextual information (role in the process, upstream/downstream dependencies). Edges are similarly enriched, capturing the nature of the streams—material, energy, or information—their directionality, composition, and any relevant process conditions. This semantic enrichment ensures that the graph is not merely a structural map, but a fully interpretable model of the chemical process, suitable for both symbolic reasoning and neural inference.

The novelty of the domain-specific graph encoder lies in its ability to bridge the gap between human-centric process design and machine-driven automation. By embedding chemical engineering ontologies, rule sets, and physical laws directly into the graph structure, the encoder enables large language models (LLMs) and symbolic reasoners to operate on process graphs with a level of understanding and fidelity previously unattainable. For example, the encoder can enforce mass and energy conservation across the network, validate the logical consistency of module interconnections, and flag potential violations of thermodynamic or safety constraints. It can also infer implicit relationships, such as recycle loops or control hierarchies, and encode these as higher-order graph constructs. This deep semantic encoding allows downstream modules—such as the infrastructure generator 320 or the symbolic validator 316—to reason over the process at multiple levels of abstraction, from high-level flows to detailed device configurations.

Furthermore, the domain-specific graph encoder 314 is designed for extensibility and interoperability. It supports the integration of new module types, process patterns, and domain rules without requiring changes to its core logic. This is achieved through a modular architecture, where domain ontologies and constraint libraries can be dynamically loaded and applied to the graph encoding process. The encoder can also interface with external data sources, simulation engines, or control systems, embedding real-time data or historical performance metrics into the graph as additional features. This flexibility ensures that the encoded graphs remain relevant and actionable across a wide range of chemical engineering applications, from batch process optimization to continuous plant control.

By providing a structured, interpretable, and semantically rich representation of chemical processes, the domain-specific graph encoder 314 empowers the neuro-symbolic reasoner 310 to perform advanced tasks such as automated model selection, error detection, and workflow orchestration. It enables LLMs to generate executable code, infrastructure-as-code artifacts, and simulation scripts that faithfully reflect the intent and constraints of the process designer. At the same time, it allows symbolic reasoners to validate, explain, and audit simulation decisions, ensuring scientific rigor and traceability. In essence, the domain-specific graph encoder transforms raw process topologies into actionable knowledge graphs, laying the foundation for intelligent, scalable, and explainable chemical engineering simulation in distributed, cloud-native environments.

The symbolic validator 316 is designed to ensure that chemical process configurations and simulation workflows adhere to rigorous scientific, logical, and domain-specific constraints. The symbolic validator 316 may operate as a deterministic and explainable layer atop the generative outputs of large language models (LLMs). The symbolic validator 316 may leverage formal rule sets, chemical engineering ontologies, and encoded physical laws to systematically validate the integrity and feasibility of process representations. This module bridges the gap between the flexible, adaptive reasoning of neural networks and the strict requirements of chemical engineering practice, providing a safeguard against errors, inconsistencies, and violations of fundamental principles.

The symbolic validator 316 ingests structured process graphs, XML schemas, or other machine-readable representations produced by upstream modules such as the process topology extractor 312 and the domain-specific graph encoder 314. It parses these inputs to identify all unit operations, streams, parameters, and interconnections, then applies a suite of validation routines tailored to chemical engineering systems. These routines include checks for mass and energy conservation, proper connectivity between modules, valid parameter ranges, and compliance with thermodynamic and safety constraints. For example, the validator can ensure that the sum of input and output flows for each reactor or separator is balanced, that temperature and pressure values fall within safe operating limits, and that no circular dependencies or disconnected modules exist within the process topology.

A key innovation of the symbolic validator is its use of domain ontologies and rule-based logic to interpret and enforce scientific constraints. Rather than relying solely on hard-coded checks, the validator references a knowledge base of chemical engineering concepts, device types, and process relationships. This enables it to reason about the intended function of each module, infer implicit connections, and detect subtle errors that may arise from incomplete or ambiguous user input. For instance, if a process configuration specifies a compressor feeding directly into a membrane separation unit, but the pressure parameters for the membrane are set below the minimum required for effective separation, the symbolic validator can detect this inconsistency, explain that the membrane unit requires a higher feed pressure to operate correctly, and suggest adjusting the compressor output or membrane parameters to ensure valid simulation results. Similarly, it can identify parameter mismatches —such as incompatible flow rates or pressure drops between connected modules—and provide detailed, traceable explanations for each validation outcome.

The symbolic validator 316 may be integrated with other components of the environment 300, supporting both pre-deployment and runtime validation. During the design phase, it may work in concert with the graphical user interface (GUI) 302 and the diagram-to-graph converter 304 to provide real-time feedback to users, highlighting errors, missing parameters, or logical inconsistencies as process diagrams are constructed. This interactive validation helps users iteratively refine their designs, reducing the risk of costly mistakes and ensuring that only scientifically sound configurations proceed to simulation or deployment. Once a process graph is finalized, the validator 316 may collaborate with the graph-to-IAC converter 306 to verify that the generated infrastructure-as-code artifacts faithfully implement the intended process logic, with all dependencies and constraints properly resolved.

Beyond static validation, the symbolic validator 316 supports dynamic, feedback-driven workflows by monitoring simulation execution and post-processing results. It can analyze output data from distributed compute resources, check for violations of process constraints during runtime, and trigger alerts or corrective actions if anomalies are detected. This capability is particularly valuable in cloud-native, event-driven environments, where simulations may be scaled, parallelized, or adapted on the fly. By maintaining a comprehensive record of validation decisions and their scientific justifications, the symbolic validator 316 ensures auditability and traceability throughout the simulation lifecycle, supporting regulatory compliance and facilitating collaboration among engineers, scientists, and stakeholders.

In summary, the symbolic validator 316 embodies a novel approach to process validation in chemical engineering simulation, combining the flexibility of neuro-symbolic AI with the rigor of formal scientific reasoning. Its deterministic, explainable validation routines provide a robust foundation for reliable, scalable, and transparent simulation workflows, enabling advanced automation while preserving the highest standards of scientific fidelity. By integrating seamlessly with other modules in the environment 300, the symbolic validator 316 transforms the neuro-symbolic reasoner 310 into a trustworthy orchestrator of complex chemical processes, capable of bridging human expertise and computational execution in distributed, cloud-native platforms.

The automated model selector 318 is designed to intelligently and autonomously determine the most appropriate mathematical or computational model for each process module in a chemical engineering simulation. This submodule leverages both neural inference—drawing on the generalization and pattern recognition capabilities of large language models (LLMs)—and symbolic reasoning, which enforces domain-specific rules, ontologies, and physical constraints. The selector operates in a context-aware manner, taking into account the specific configuration of the process topology, the quality and completeness of available data, and the explicit or implicit goals set by the user, such as accuracy, computational efficiency, or robustness to uncertainty.

The automated model selector 318 includes a hybrid decision engine that continuously evaluates the simulation context. When a process topology is parsed and encoded by upstream modules such as the process topology extractor 312 and the domain-specific graph encoder 314, the selector 318 receives a richly annotated graph representation of the chemical process. Each node in this graph corresponds to a process module—such as a reactor, separator, compressor, or membrane—and is tagged with metadata including device type, operating parameters, available sensor data, and any user-specified preferences or constraints. The selector 318 analyzes this information to determine which mathematical models are available for each module, ranging from first-principles, physics-based equations to empirical correlations or surrogate models.

The novelty of the automated model selector 318 lies in its ability to reason across multiple layers of abstraction. Using neural components, the selector 318 can interpret ambiguous or incomplete user input, infer likely modeling requirements from historical data or prior simulations, and generalize to novel process configurations. For example, if a user provides only partial information about a membrane unit, the selector 318 can draw on learned patterns to propose a suitable model, estimate missing parameters, or suggest default values. At the same time, the symbolic reasoning layer ensures that any proposed model adheres to fundamental scientific principles—such as mass and energy conservation, thermodynamic feasibility, and process safety limits—by referencing encoded domain ontologies and rule sets. This dual approach allows the selector 318 to blend or switch between models dynamically, adapting to changes in process conditions, data availability, or user objectives.

The automated model selector 318 may be integrated with other modules in the environment 300. It may collaborate with the symbolic validator 316 to ensure that selected models are logically consistent with the overall process configuration and that all required parameters are specified or inferable. When the simulation is executed and new data becomes available—such as real-time sensor readings or intermediate results—the selector 318 can trigger model updates or refinements, enabling feedback-driven optimization and continuous improvement of simulation fidelity. In advanced embodiments, the selector 318 may support model blending, where multiple candidate models are combined—using techniques such as ensemble learning, weighted averaging, or context-dependent switching—to achieve optimal trade-offs between accuracy, speed, and interpretability.

By automating the traditionally manual and expertise-intensive task of model selection, the automated model selector 318 significantly reduces the burden on users, improves access to advanced simulation capabilities, and ensures that each process module is represented by the most suitable model for the task at hand. This context-aware, adaptive approach not only accelerates the simulation workflow but also enhances the reliability, transparency, and scientific rigor of distributed chemical engineering simulations. The selector's ability to operate across modalities—integrating graphical, textual, and structured data—and to reason both statistically and symbolically, represents a substantial advancement over conventional, static simulation platforms.

The infrastructure generator 320 is responsible for translating high-level, semantically rich process representations into concrete, executable artifacts that drive both simulation and deployment. Building upon the outputs of upstream modules—such as the process topology extractor 312 and the domain-specific graph encoder 314—the infrastructure generator 320 ingests a fully annotated process graph that encodes the topology, parameters, and interconnections of a chemical engineering workflow. Leveraging both neural and symbolic reasoning, this submodule synthesizes a range of output modalities, including simulation code (for example, Python scripts, MATLAB routines, or domain-specific simulation kernels) and infrastructure-as-code (IaC) deployment scripts (such as Terraform, AWS CloudFormation, or Pulumi templates).

The infrastructure generator 320 bridges the gap between abstract process design and practical execution environments. Rather than requiring manual scripting or ad hoc configuration, the generator 320 interprets the process graph as a set of modular, composable components—each corresponding to a unit operation, stream, or control element —and maps these to computational resources in a cloud-native or hybrid infrastructure. For instance, a reactor node in the process graph may be translated into a Python class implementing the relevant reaction kinetics, while simultaneously being mapped to a serverless function or containerized microservice in the IaC template. The generator 320 encodes not only the logic of each module but also the orchestration of data flows, dependencies, and execution order, ensuring that the deployed infrastructure faithfully mirrors the intended process topology.

The infrastructure generator 320 is designed for extensibility and adaptability. It supports multiple output targets, allowing users to generate simulation code for local execution, cloud-based workflows, or integration with third-party simulation engines. The generator 320 can produce workflow definitions for serverless orchestration platforms (such as AWS Step Functions or Azure Durable Functions), encode message-passing interfaces using cloud-native queues or event buses, and generate configuration files for monitoring, logging, and error handling. By reasoning over the semantic annotations in the process graph, the generator can tailor resource allocation, optimize deployment strategies, and enforce compliance with user-specified constraints such as cost, performance, or security.

The infrastructure generator 320 may collaborate with the symbolic validator 316 to ensure that generated artifacts are logically consistent and scientifically sound, performing pre-deployment checks for parameter completeness, connectivity, and constraint satisfaction. It also interfaces with the agent orchestrator 322, providing the executable artifacts and deployment plans required for distributed simulation and real-time task management. In advanced embodiments, the generator supports round-trip engineering, allowing changes in the deployed infrastructure to be reflected back into the process graph, thus enabling iterative design and continuous improvement.

By automating the translation from high-level, human-centric process design to low-level, machine-executable code and infrastructure, the infrastructure generator 320 fundamentally accelerates the simulation lifecycle. It empowers engineers to focus on process innovation and optimization, while ensuring that the underlying computational and deployment complexities are handled transparently and robustly. This capability not only enhances reproducibility and scalability but also supports a wide range of use cases—from rapid prototyping and parameter sweeps to large-scale, production-grade chemical process simulations in distributed, cloud-native environments. The infrastructure generator 320 thus provides a seamless, intelligent, and scientifically rigorous pipeline from conceptual process design to automated, scalable execution.

The agent orchestrator 322 serves as the central nervous system of the neuro-symbolic reasoner 310, enabling the intelligent, dynamic coordination of distributed simulation workflows in chemical engineering environments. The agent orchestrator 322 is responsible for decomposing complex, high-level simulation objectives into modular, manageable tasks, each of which can be delegated to specialized agents—whether they are large language models (LLMs), symbolic reasoning modules, or hybrid neuro-symbolic subcomponents. This decomposition is not static; rather, the orchestrator 322 continuously analyzes the evolving state of the simulation, adapting the task graph in real time to accommodate new data, user interventions, or emergent system behaviors.

The agent orchestrator 322 leverages a multi-agent architecture inspired by frameworks such as LangGraph, where each node in the orchestration graph represents a distinct agent with domain-specific expertise. For example, one agent may specialize in parsing and encoding process topologies from graphical user interfaces, while another may focus on translating symbolic representations into infrastructure-as-code (IaC) artifacts, and yet another may handle the validation of physical and logical constraints. The orchestrator 322 manages the flow of information between these agents using structured communication protocols, ensuring that messages—such as simulation parameters, intermediate results, or error reports—are routed efficiently and semantically correctly. This structured messaging maintains consistency and traceability across the distributed workflow, especially as tasks are executed in parallel or iteratively refined.

The agent orchestrator 322 supports dynamic, feedback-driven simulation and optimization. Unlike traditional, linear simulation pipelines, the orchestrator 322 enables closed-loop workflows where the outputs of one agent can trigger the reconfiguration or re-execution of upstream tasks. For instance, if the feedback engine 324 detects that a simulation result violates a process constraint or fails to meet a user-defined objective, the orchestrator 211 can automatically invoke the symbolic validator 316 to diagnose the issue, prompt the automated model selector 318 to propose alternative models, or instruct the infrastructure generator 320 to adjust deployment parameters. This feedback-driven approach supports both forward problem solving—where the goal is to predict system behavior given a process configuration—and inverse problem solving, where the system must infer optimal process parameters or configurations to achieve a desired outcome. The orchestrator's ability to manage these iterative, multi-directional workflows allows for advanced applications such as process optimization, design-space exploration, and adaptive control.

The agent orchestrator 322 is designed for extensibility and interoperability. It can integrate new agent types or reasoning modules as the simulation environment evolves, allowing the system to scale in both breadth and depth of capability. By maintaining a comprehensive execution trace and supporting standardized interfaces, the orchestrator ensures that each agent's contributions are auditable and that the overall workflow remains robust in the face of partial failures or unexpected inputs. Furthermore, the orchestrator 322 can interface with external systems—such as cloud orchestration platforms, simulation engines, or enterprise data sources—enabling seamless integration of the neuro-symbolic reasoner 310 into broader chemical engineering IT ecosystems.

In summary, the agent orchestrator 322 transforms the neuro-symbolic reasoner from a collection of isolated AI and symbolic modules into a cohesive, adaptive, and intelligent system. By decomposing complex workflows, coordinating specialized agents, and managing structured communication and feedback, it enables a new level of automation, flexibility, and scientific rigor in distributed chemical engineering simulation. This orchestration capability helps to realize dynamic, explainable, and scalable simulation platforms that bridge the gap between domain expertise and computational execution.

The feedback engine 324 is designed to enable intelligent, adaptive, and closed-loop optimization of chemical engineering simulations in distributed, cloud-native environments. Its primary function is to analyze the results generated by simulation workflows—whether those results are raw numerical outputs, process performance metrics, or diagnostic logs—and to use this information to drive iterative refinement of process parameters, module configurations, and even the underlying simulation topology. By integrating both neural and symbolic reasoning, the feedback engine 324 transforms the simulation environment from a static, one-shot execution model into a dynamic, learning-oriented system capable of continuous improvement and self-optimization. In certain embodiments, the feedback engine 324 may employ reinforcement learning techniques to systematically explore and optimize process configurations, using simulation outcomes as feedback signals to guide the selection of parameter adjustments and control strategies over successive iterations. This approach enables the system to autonomously discover improved solutions and adapt to changing process requirements in real time.

At the heart of the feedback engine 324 is its ability to interpret simulation outcomes in the context of user-defined objectives, domain-specific constraints, and historical performance data. Upon completion of a simulation run, the engine ingests output data from distributed compute resources—such as AWS Lambda functions, containers, or virtual machines—aggregating results across multiple modules and simulation instances. It applies statistical analysis, trend detection, and anomaly identification to assess whether the simulation has met its goals, such as maximizing yield, minimizing energy consumption, or achieving target product specifications. When deviations from desired outcomes are detected, the feedback engine leverages AI-driven techniques—including reinforcement learning, Bayesian optimization, or evolutionary algorithms—to propose adjustments to process parameters, such as flow rates, temperatures, pressures, or module selection.

The feedback engine 324 integrates symbolic reasoning with data-driven optimization. While neural components can suggest parameter updates based on observed trends or learned patterns, the symbolic layer ensures that any proposed changes remain consistent with physical laws, process safety limits, and domain ontologies. For example, if the engine 324 identifies that increasing reactor temperature could improve conversion, the symbolic validator 316 is invoked to check that the new temperature remains within safe operating bounds and does not violate material constraints. This hybrid approach ensures that optimization is not only effective but also scientifically and operationally sound.

The feedback engine 324 may interface with the agent orchestrator 322 to trigger re-deployment of updated simulation workflows, ensuring that new parameter sets or module configurations are propagated throughout the distributed system. The feedback engine 324 may collaborate with the automated model selector 318 to determine whether alternative mathematical models or surrogate approximators should be employed in subsequent simulation cycles, especially when discrepancies between predicted and observed results are detected. The engine 324 also may maintain a comprehensive traceability record, documenting each iteration's parameter choices, simulation outcomes, and optimization decisions, thereby supporting auditability and regulatory compliance.

In advanced embodiments, the feedback engine 324 supports multi-objective and multi-modal optimization, balancing competing goals such as cost, efficiency, and environmental impact. It can orchestrate large-scale parameter sweeps, design-of-experiments studies, or adaptive sampling campaigns, leveraging the scalability of cloud infrastructure to explore vast design spaces efficiently. The engine's closed-loop architecture enables real-time adaptation: as new data becomes available—whether from simulation, experimental validation, or process monitoring—the engine updates its optimization strategy, refines its models, and iteratively converges toward optimal process configurations.

By embedding AI-driven feedback and optimization directly into the simulation lifecycle, the feedback engine 324 fundamentally enhances the agility, intelligence, and robustness of chemical engineering workflows. It empowers users to move beyond static, trial-and-error approaches, enabling automated discovery of optimal process designs, rapid response to changing requirements, and continuous improvement of simulation fidelity. This capability is important for modern, data-intensive chemical engineering, where the complexity of process systems and the scale of computational resources demand adaptive, self-improving solutions that bridge the gap between domain expertise and computational execution. In particular, AI-driven feedback not only supports iterative process design and optimization, but also enables real-time automation control, allowing chemical processes to be dynamically adjusted and managed as operating conditions evolve.

The traceability module 326 within the neuro-symbolic reasoner 310 is designed to provide comprehensive explainability and end-to-end traceability throughout the lifecycle of distributed chemical engineering simulations. This module operates as a persistent, intelligent ledger that records every significant transformation, decision, and artifact generated by the system, from the initial user input through to the final simulation results and infrastructure deployment. Its core function is to capture step-by-step derivations and validation traces, ensuring that every action taken by the neuro-symbolic reasoner—whether neural, symbolic, or hybrid—can be reconstructed, audited, and explained in detail.

The traceability module 326 logs semantic explanations for each transformation and decision. As the system ingests a graphical process diagram or a natural language description, the traceability module 326 records how the process topology extractor 312 interprets and encodes the input, including the rationale for identifying specific modules, connections, and parameters. When the domain-specific graph encoder 314 enriches the process graph with chemical semantics and metadata, the traceability module 326 documents the ontologies and rule sets applied, as well as any inferred relationships or constraint checks. As the symbolic validator 316 performs logical and scientific validation, the module 326 logs each rule invoked, the outcome of every constraint check, and the justification for any flagged errors or warnings. This level of detail extends to the automated model selector 318, where the traceability module 326 records the context, criteria, and reasoning behind model selection or blending, including fallback strategies and parameter estimation logic.

When the infrastructure generator 320 translates validated process graphs into infrastructure-as-code artifacts or simulation scripts, the traceability module 326 maintains a mapping between the original process representation and the generated code or configuration. This mapping includes not only the technical details of resource allocation and orchestration, but also the semantic intent—such as why a particular module was mapped to a serverless function versus a container, or how message-passing interfaces were structured to reflect process flows. As the agent orchestrator 322 coordinates distributed execution, the traceability module 326 tracks the flow of messages, the invocation of agents, and the resolution of feedback loops, capturing both the high-level workflow and the granular execution steps.

The traceability module 326 supports bidirectional traceability and explainability. This explainability is primarily assured by the symbolic reasoning components, which provide transparent, rule-based logic and detailed validation traces that can be reviewed and audited, in contrast to the neural components that may operate as opaque models. Not only does it allow users, auditors, or regulators to reconstruct the provenance of any simulation result or infrastructure deployment, but it also enables reverse mapping from outcomes back to the originating process design and decision logic. For example, if a simulation output is questioned or a regulatory inquiry arises, the traceability module 326 can provide a detailed, stepwise account of how the result was produced, including the data sources, model choices, validation checks, and infrastructure mappings involved at each stage. This capability allows for regulatory compliance, quality assurance, and scientific reproducibility, particularly in safety-critical or highly regulated chemical engineering domains.

The traceability module 326 may interface with the feedback engine 324, enabling closed-loop optimization and continuous improvement. As simulation results are analyzed and iterative refinements are proposed, the module 326 records each feedback cycle, the parameter adjustments made, and the justifications for those changes. This creates a living history of the simulation lifecycle, supporting both retrospective analysis and proactive optimization.

By embedding explainability and traceability as first-class features of the neuro-symbolic reasoner 310, the traceability module 326 ensures that advanced AI-driven automation does not come at the expense of transparency or control. The traceability module 326 transforms the system from a black box into an auditable, scientifically rigorous platform, where every decision and transformation is documented, justified, and accessible for review. This not only builds trust with users and stakeholders but also establishes a robust foundation for regulatory approval, collaborative engineering, and the long-term evolution of chemical simulation workflows.

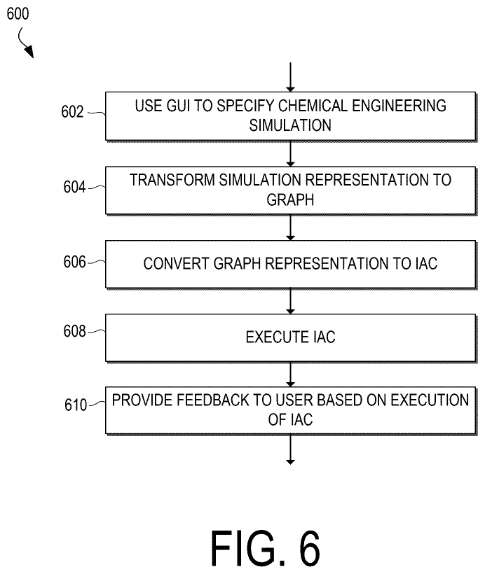

The method 600 illustrated in FIG. 6 provides a systematic approach for automating chemical engineering simulations using cloud-native infrastructure and optionally leveraging AI/neuro-symbolic reasoning.

In block 602, in an initial step, a graphical user interface (GUI) is used to allow users—such as chemical engineers or process designers—to visually construct and configure a chemical engineering simulation. The GUI typically presents a palette of process modules (e.g., reactors, compressors, heat exchangers, separators) that can be dragged and dropped onto a workspace. Users connect these modules with streams or channels representing the flow of materials, energy, or information. Each module and connection can be parameterized with relevant operating conditions, such as temperature, pressure, flow rate, and composition. The GUI is designed to be intuitive and interactive, supporting real-time validation, configuration panels, and import/export of process diagrams in structured formats y (such as XML or JSON). While this step can be implemented entirely with conventional software, an AI/neuro-symbolic reasoner may be used to assist users by suggesting module arrangements, validating logical consistency, or interpreting natural language descriptions of the process.

In block 604, once the process diagram is constructed in the GUI, the next step involves transforming this visual representation into a structured graph model, which may be encoded in, e.g., XML, JSON, etc. This graph encodes the topology of the chemical process, with nodes representing unit operations and edges representing streams or connections. The transformation can be performed by parsing the GUI's internal data structures or by converting exported files (e.g., XML, JSON) into a formal graph schema. The resulting graph includes metadata for each module (type, parameters, unique identifiers) and for each connection (direction, stream properties). This step is essential for enabling downstream automation and integration with computational workflows. AI/neuro-symbolic modules can enhance this step by inferring missing connections, validating the graph against domain-specific rules, or translating ambiguous user input into precise graph elements. In an illustrative embodiment, the output is an IAC, which may represented as XML or Terraform code

In block 606, the structured graph is then converted into infrastructure-as-code (IAC) artifacts, such as Terraform or CloudFormation templates. This conversion maps each process module to a corresponding computational resource (e.g., serverless function, container, virtual machine) and encodes the data flows between modules as message queues, event triggers, or network connections. The IAC artifacts specify the provisioning, configuration, and orchestration of resources in a cloud environment, ensuring that the deployed infrastructure mirrors the logical process graph. This step can be performed using rule-based mapping logic or template generators. Optionally, an AI/neuro-symbolic reasoner can automate the generation of IAC templates, optimize resource allocation, and ensure that the infrastructure configuration adheres to scientific and operational constraints.

In block 608, with the IAC artifacts generated, the next step is to execute them —provisioning the defined cloud resources and deploying the simulation components. The execution involves creating compute instances, serverless functions, message queues, and other infrastructure elements as specified in the IAC templates. Simulation tasks are scheduled and run across the distributed resources, with data flowing between modules according to the process topology. The execution is managed by orchestration tools (e.g., Terraform, AWS Step Functions) and may include monitoring, error handling, and dynamic scaling. While this step is primarily a technical deployment process, AI/neuro-symbolic modules can be integrated to monitor execution, detect anomalies, and adapt resource allocation in real time.

In block 610, after the simulation is executed, feedback is provided to the user. This feedback may include simulation results, performance metrics, diagnostic information, and visualizations of process flows. The system aggregates output data from the distributed resources, performs post-processing (such as validation, statistical analysis, or transformation into user-friendly formats), and presents the results through the GUI. Users can review detailed reports, inspect module-specific outputs, and iteratively refine their process design based on the feedback. AI/neuro-symbolic reasoners can further enhance this step by analyzing results, suggesting optimizations, detecting errors, and enabling closed-loop revision cycles—where user adjustments trigger re-deployment and re-execution of the simulation.

In summary, method 600 provides a robust framework for automating chemical engineering simulations using cloud infrastructure, with each step supporting both conventional and AI-augmented implementations. The integration of neuro-symbolic reasoning enables intelligent orchestration, validation, and optimization throughout the simulation lifecycle, while the core workflow remains accessible and effective for users regardless of AI involvement.

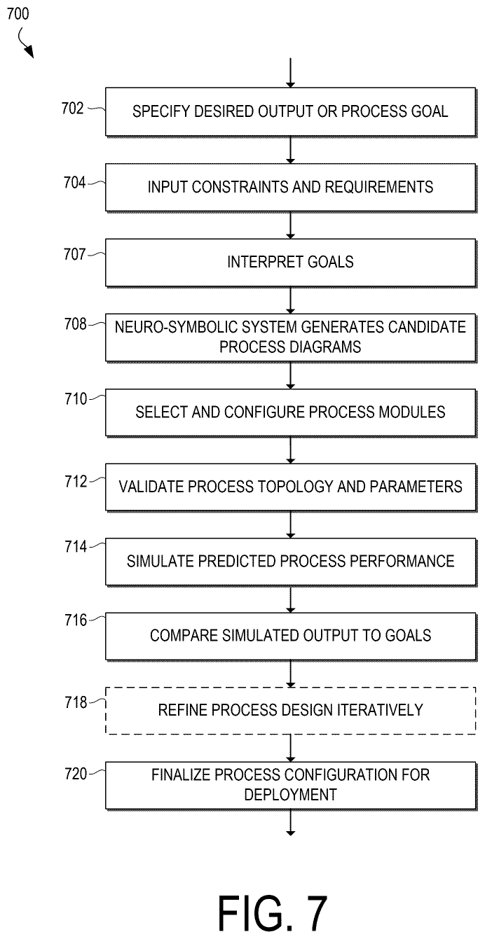

Referring now to FIG. 7, in one embodiment, method 700 provides a comprehensive, AI-augmented framework for automating chemical process simulation and design using a hybrid neuro-symbolic multi-agent system. This method leverages advanced artificial intelligence—including large language models (LLMs) and neuro-symbolic reasoning—to interpret user goals, generate and validate process configurations, simulate performance, and iteratively optimize the process design. The workflow is modular and adaptive, supporting both conventional and AI-driven implementations, and is particularly suited for deployment in distributed, cloud-native environments.

The method begins in block 702, with the user or enterprise system specifying the desired output or process goal. This input may take the form of a target product specification, a set of performance metrics (such as yield, purity, or energy efficiency), or a high-level operational objective (e.g., minimize waste, maximize throughput). The process goal can be provided through a graphical user interface, a structured file (such as XML or JSON), or even a natural language description. The system is designed to accommodate a wide range of input modalities, ensuring that user intent is captured accurately and flexibly.

In block 704, the user or system provides any relevant constraints and requirements that must be satisfied by the process. These may include physical, safety, regulatory, or economic constraints—such as maximum allowable pressure, temperature limits, environmental regulations, or cost ceilings. The constraints can be specified explicitly (e.g., as parameter ranges or logical conditions) or inferred from domain knowledge and historical data. This step ensures that the subsequent process design and simulation are grounded in real-world feasibility and compliance.

In block 706, the neuro-symbolic system interprets the specified goals and constraints, translating them into actionable process requirements. Advanced LLMs and symbolic reasoning modules parse the input—whether graphical, structured, or textual—and extract the underlying process objectives, key performance indicators, and operational boundaries. The system may leverage domain ontologies and knowledge graphs to contextualize the goals, ensuring that the intent is faithfully mapped to technical specifications. This interpretation forms the foundation for automated process synthesis and optimization.

In block 708, leveraging its hybrid AI capabilities, the neuro-symbolic system generates one or more candidate process diagrams that could achieve the specified goals within the given constraints. LLMs are used to propose process topologies, select appropriate unit operations, and suggest interconnections based on learned patterns and domain expertise. Symbolic reasoning modules ensure that the generated diagrams are logically consistent and adhere to engineering principles. This step may involve the creation of multiple alternative configurations, each representing a different approach to meeting the process objectives.

In block 710, once candidate process diagrams are generated, the system proceeds to select and configure the individual process modules (e.g., reactors, separators, heat exchangers) that comprise the process. The neuro-symbolic system draws on a library of available module types and models, using both neural inference and symbolic rules to determine the most suitable options for each role in the process. Module parameters—such as size, operating conditions, and control strategies—are configured based on the process requirements, available data, and optimization criteria. This step ensures that each module is tailored to the specific needs of the overall process.

In block 712, the configured process topology and parameters are then subjected to rigorous validation. Symbolic validators apply domain-specific rules, physical laws (such as mass and energy balances), and safety constraints to check for logical consistency, completeness, and feasibility. The neuro-symbolic system can detect errors, omissions, or parameter mismatches, providing detailed feedback and suggestions for correction. This validation step is critical for ensuring that the process design is scientifically sound and ready for simulation.

In block 714, with a validated process configuration, the system simulates the predicted performance of the process using appropriate mathematical and computational models. The simulation may be executed locally or distributed across cloud resources, depending on the complexity and scale of the process. The neuro-symbolic system can automate model selection, orchestrate simulation workflows, and aggregate results. Simulation outputs include key performance metrics, process variable profiles, and diagnostic information, providing a quantitative basis for evaluating the process design.

In block 716, the results of the simulation are compared to the original process goals and constraints. The neuro-symbolic system analyzes the degree to which the simulated outputs meet the specified objectives, identifying any gaps or areas for improvement. This comparison may involve statistical analysis, trend detection, and multi-objective optimization, leveraging both AI-driven and rule-based techniques. If the process fails to meet the goals, the system identifies the root causes and prioritizes areas for refinement.

In block 718, in some embodiments, based on the comparison, the system may enter an iterative refinement loop. The neuro-symbolic system proposes adjustments to the process topology, module selection, or parameter values, guided by optimization algorithms and domain knowledge. Each iteration involves re-validation and re-simulation, with the system learning from previous results to converge toward an optimal or satisfactory process configuration. This closed-loop, feedback-driven approach enables rapid prototyping, continuous improvement, and adaptive optimization.

In block 720, once the process design meets all goals and constraints, the system finalizes the configuration for deployment. This may include generating infrastructure-as-code artifacts, workflow definitions, and deployment scripts for cloud or on-premises execution. The neuro-symbolic system ensures that the final process configuration is fully documented, traceable, and ready for implementation. Users receive comprehensive reports, visualizations, and deployment instructions, enabling seamless transition from design to operation.

The described neuro-symbolic system demonstrates that large language models (LLMs), when integrated with symbolic reasoning and domain-specific knowledge, can do far more than merely translate a pre-defined technical specification into simulation infrastructure—they can actively invent novel chemical processes from scratch. Given only a high-level objective, such as “produce X kilograms per hour of chemical Y with minimal environmental impact,” the system autonomously generates candidate process diagrams by synthesizing known unit operations (e.g., reactors, separators, heat exchangers) into coherent flowsheets. This “online creation” capability stems from the LLM's ability to reason over a vast library of process modules, guided by both learned patterns from training data and hard-coded physical and safety constraints. Rather than acting as a passive interpreter, the model functions as a creative co-engineer, proposing plausible, physically consistent process architectures that fulfill the target specification.

This generative capacity is significantly enhanced by the closed-loop, iterative refinement architecture outlined in blocks 710-720. After an initial process is proposed, the system validates it against mass and energy balances, simulates its performance, compares outcomes to design goals, and—crucially—learns from discrepancies to propose improved alternatives. The LLM, embedded within this neuro-symbolic framework, leverages both data-driven intuition and rule-based rigor to navigate the vast design space efficiently, converging on feasible, optimized solutions without human intervention. Thus, the proposed LLM technology transcends traditional automation by enabling autonomous process creation: it doesn't just implement instructions—it conceives, tests, and refines chemical manufacturing strategies in response to abstract production goals, dramatically accelerating innovation in process engineering.

In summary, method 700 provides an intelligent, modular, and explainable workflow for chemical process simulation and design, integrating advanced AI and neuro-symbolic reasoning at every stage to maximize automation, scientific rigor, and user accessibility.

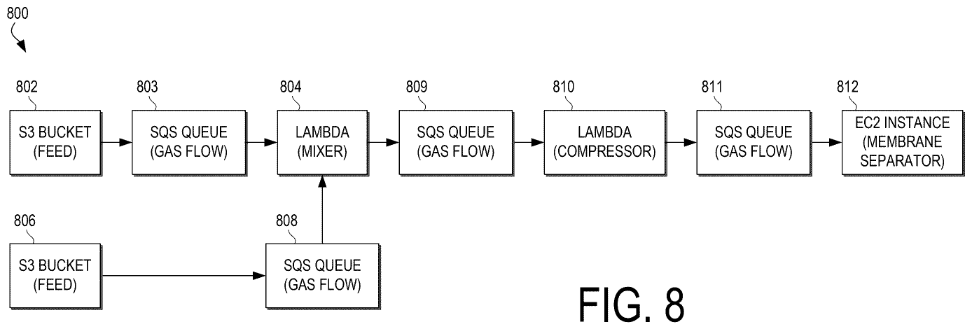

Referring now to FIG. 8, in one embodiment, a data flow 800 for a cloud-based chemical engineering simulation system is shown, which corresponds to the graphical process modules depicted in FIG. 4. Components in FIG. 8 are mapped to their functional counterparts in FIG. 4, illustrating how the graphical user interface and process diagram are translated into a distributed, event-driven computational workflow.

The input layer in FIG. 8 consists of two S3 buckets: S3 Bucket 802 and S3 Bucket 806. S3 Bucket 802 corresponds to the reagent source 406 in FIG. 4, while S3 Bucket 806 maps to reagent source 408. These buckets serve as the initial data repositories, storing incoming Excel files that contain the process input parameters and reagent data. When a file is uploaded to either bucket, S3 automatically generates an event notification, which initiates the downstream processing sequence.

Central to the orchestration of data flow is a message-passing fabric implemented with SQS queues. In FIG. 8, SQS queue 803 receives messages originating from S3 Bucket 802, and SQS queue 808 receives messages from S3 Bucket 806. These queues decouple the input layer from the first processing stage. After mixing, SQS queue 809 buffers the mixed gas stream prior to compression, and SQS queue 811 buffers the compressed gas stream prior to membrane separation. Collectively, the SQS queues 803, 808, 809, and 811 provide reliable delivery, backpressure, and retry semantics between adjacent stages of the workflow, ensuring robust and scalable operation.

The processing layer is implemented using two Lambda functions: Lambda Mixer 804 and Lambda Compressor 810. Lambda Mixer 804 in FIG. 8 is mapped to the mixer 410 in FIG. 4. This function reads messages from SQS queues 803 and 808, waits until data from both feeds is available, and then combines or mixes the input data, replicating the mixing operation performed by mixer 410 in the graphical process diagram. The mixed output is published to SQS queue 809. Lambda Compressor 810 in FIG. 8 corresponds to compressor 412 in FIG. 4. This function is triggered by messages on SQS queue 809 and performs the compressing operation, after which the compressed stream is emitted to SQS queue 811. Both Lambda functions operate in a serverless manner, scaling automatically and running only when needed.

Once the data has been mixed and compressed, it is sent to the compute layer, represented by the EC2 instance 812 in FIG. 8. The EC2 instance 812, labeled as a membrane separator in FIG. 8, corresponds to the membrane unit 414 in FIG. 4. It consumes the compressed gas stream from SQS queue 811 and performs the membrane separation computations, such as modeling permeate and retentate streams under the specified operating conditions for the separation task.

In summary, the data flow begins with the upload of Excel files to S3 Bucket 802 (reagent source 406) and S3 Bucket 806 (reagent source 408). S3 event notifications place messages onto SQS queues 803 and 808, which feed Lambda Mixer 804 (mixer 410). The mixer combines data from both inputs and publishes the mixed dataset to SQS queue 809, which triggers Lambda Compressor 810 (compressor 412). The compressor outputs a compressed stream to SQS queue 811, and that stream is delivered to EC2 instance 812, which implements the membrane separator (membrane unit 414). This architecture faithfully mirrors the process topology of FIG. 4, translating each graphical module into a corresponding cloud-native component with intermediate gas-flow queues that decouple stages and enable automated, scalable chemical engineering simulation.

### EXAMPLES

Illustrative examples of the technologies disclosed herein are provided below. An embodiment of the technologies may include any one or more, and any combination of, the examples described below.

Example 1 includes a compute device for automating chemical engineering simulations, the compute device comprising a processor; memory coupled to the processor; one or more computer readable media comprising a plurality of instructions stored thereon that, when executed by the processor, cause the processor to provide a graphical user interface (GUI) for constructing and configuring a chemical process simulation; transform a process diagram created in the GUI into a structured graph model representing the process diagram; convert the structured graph model into infrastructure-as-code (IaC) artifacts for deployment in a cloud environment; and execute the IaC artifacts to provision and deploy simulation components in the cloud environment.

Example 2 includes the subject matter of Example 1, and wherein the plurality of instructions further cause the compute device to provide one or more simulation results to a user on the GUI.

Example 3 includes the subject matter of any of Examples 1 and 2, and wherein the structured graph model comprises a machine-readable representation selected from the group consisting of XML, JSON, or a domain-specific schema.

Example 4 includes the subject matter of any of Examples 1-3, and wherein to convert the structured graph model into infrastructure-as-code (IaC) artifacts comprises to generate one or more templates selected from the group consisting of Terraform, AWS CloudFormation, Pulumi, or Ansible templates.

Example 5 includes the subject matter of any of Examples 1-4, and wherein the simulation components are deployed as one or more of serverless functions, containers, or virtual machines in the cloud environment.

Example 6 includes the subject matter of any of Examples 1-5, and wherein the process diagram comprises a plurality of process modules selected from the group consisting of reactors, compressors, heat exchangers, separators, mixers, membranes, pumps valves, filters, distillation columns, absorption columns, extraction units, centrifuges, decanters, crystallizers, evaporators, condensors, and other chemical engineering modules.

Example 7 includes the subject matter of any of Examples 1-6, and wherein the GUI enables a user to specify parameters for each process module input flow, including at least one of flow rate, pressure, temperature, or composition.

Example 8 includes the subject matter of any of Examples 1-7, and wherein the structured graph model encodes a topology, parameters, and interconnections of the chemical process simulation.

Example 9 includes the subject matter of any of Examples 1-8, and wherein the simulation components communicate via message queues or events.

Example 10 includes the subject matter of any of Examples 1-9, and wherein the plurality of instructions further cause the compute device to validate the structured graph model for logical consistency, completeness, or compliance with domain-specific rules prior to converting into IaC artifacts.

Example 11 includes the subject matter of any of Examples 1-10, and wherein the plurality of instructions further cause the compute device to employ symbolic components working together with a large language model (LLM) to interpret the process diagram and assist in generating the structured graph model.

Example 12 includes the subject matter of any of Examples 1-11, and wherein the plurality of instructions further cause the compute device to use an AI agent to validate the process diagram, detect errors, or suggest corrections prior to deployment.

Example 13 includes the subject matter of any of Examples 1-12, and wherein the conversion of the structured graph model into IaC artifacts is at least partially automated by a large language model and a neuro-symbolic reasoning engine.

Example 14 includes the subject matter of any of Examples 1-13, and wherein the plurality of instructions further cause the compute device to employ an AI agent to select or configure mathematical or computational models for one or more process modules based on context or user goals.

Example 15 includes the subject matter of any of Examples 1-14, and wherein the plurality of instructions further cause the compute device to use a neuro-symbolic system to analyze simulation results and iteratively refine process parameters or configurations in a feedback-driven optimization loop.

Example 16 includes the subject matter of any of Examples 1-15, and wherein the plurality of instructions further cause the compute device to use an AI agent to translate between different modalities, including graphical user interface representations, structured files, and infrastructure-as-code artifacts.

Example 17 includes the subject matter of any of Examples 1-16, and wherein the plurality of instructions further cause the compute device to maintain a traceability record of simulation decisions, configurations, and execution outcomes for auditability and explainability.

Example 18 includes the subject matter of any of Examples 1-17, and wherein the plurality of instructions further cause the compute device to employ a large language model (LLM) to generate a chemical process configuration in response to a specified production goal for a chemical material, wherein the LLM autonomously creates a process diagram and associated parameters suitable for achieving the production goal prior to converting the process configuration into simulation infrastructure.

Example 19 includes a method for automating chemical engineering simulations, the method comprising providing, by a compute device, a graphical user interface (GUI) for constructing and configuring a chemical process simulation; transforming, by the compute device, a process diagram created in the GUI into a structured graph model representing the process diagram; converting, by the compute device, the structured graph model into infrastructure-as-code (IaC) artifacts for deployment in a cloud environment; and executing, by the compute device, the IaC artifacts to provision and deploy simulation components in the cloud environment.

Example 20 includes the subject matter of Example 19, and further including providing one or more simulation results to a user on the GUI.

Example 21 includes the subject matter of any of Examples 19 and 20, and wherein the structured graph model comprises a machine-readable representation selected from the group consisting of XML, JSON, or a domain-specific schema.

Example 22 includes the subject matter of any of Examples 19-21, and wherein converting the structured graph model into infrastructure-as-code (IaC) artifacts comprises generating one or more templates selected from the group consisting of Terraform, AWS CloudFormation, Pulumi, or Ansible templates.

Example 23 includes the subject matter of any of Examples 19-22, and wherein the simulation components are deployed as one or more of serverless functions, containers, or virtual machines in the cloud environment.

Example 24 includes the subject matter of any of Examples 19-23, and wherein the process diagram comprises a plurality of process modules selected from the group consisting of reactors, compressors, heat exchangers, separators, mixers, membranes, pumps valves, filters, distillation columns, absorption columns, extraction units, centrifuges, decanters, crystallizers, evaporators, condensors, and other chemical engineering modules.

Example 25 includes the subject matter of any of Examples 19-24, and wherein the GUI enables a user to specify parameters for each process module input flow, including at least one of flow rate, pressure, temperature, or composition.

Example 26 includes the subject matter of any of Examples 19-25, and wherein the structured graph model encodes a topology, parameters, and interconnections of the chemical process simulation.

Example 27 includes the subject matter of any of Examples 19-26, and wherein the simulation components communicate via message queues or events.

Example 28 includes the subject matter of any of Examples 19-27, and further including validating the structured graph model for logical consistency, completeness, or compliance with domain-specific rules prior to converting into IaC artifacts.

Example 29 includes the subject matter of any of Examples 19-28, and further including employing symbolic components working together with a large language model (LLM) to interpret the process diagram and assist in generating the structured graph model.

Example 30 includes the subject matter of any of Examples 19-29, and further including using an AI agent to validate the process diagram, detect errors, or suggest corrections prior to deployment.

Example 31 includes the subject matter of any of Examples 19-30, and wherein the conversion of the structured graph model into IaC artifacts is at least partially automated by a large language model and a neuro-symbolic reasoning engine.

Example 32 includes the subject matter of any of Examples 19-31, and further including employing an AI agent to select or configure mathematical or computational models for one or more process modules based on context or user goals.

Example 33 includes the subject matter of any of Examples 19-32, and further including using a neuro-symbolic system to analyze simulation results and iteratively refine process parameters or configurations in a feedback-driven optimization loop.

Example 34 includes the subject matter of any of Examples 19-33, and further including using an AI agent to translate between different modalities, including graphical user interface representations, structured files, and infrastructure-as-code artifacts.

Example 35 includes the subject matter of any of Examples 19-34, and further including maintaining a traceability record of simulation decisions, configurations, and execution outcomes for auditability and explainability.

Example 36 includes the subject matter of any of Examples 19-35, and further including employing a large language model (LLM) to generate a chemical process configuration in response to a specified production goal for a chemical material, wherein the LLM autonomously creates a process diagram and associated parameters suitable for achieving the production goal prior to converting the process configuration into simulation infrastructure.

Example 37 includes a compute device for automating chemical engineering simulations, the compute device comprising means for providing, by a compute device, a graphical user interface (GUI) for constructing and configuring a chemical process simulation; means for transforming a process diagram created in the GUI into a structured graph model representing the process diagram; means for converting the structured graph model into infrastructure-as-code (IaC) artifacts for deployment in a cloud environment; and means for executing the IaC artifacts to provision and deploy simulation components in the cloud environment.

Example 38 includes the subject matter of Example 37, and further including means for providing one or more simulation results to a user on the GUI.

Example 39 includes the subject matter of any of Examples 37 and 38, and wherein the structured graph model comprises a machine-readable representation selected from the group consisting of XML, JSON, or a domain-specific schema.

Example 40 includes the subject matter of any of Examples 37-39, and wherein the means for converting the structured graph model into infrastructure-as-code (IaC) artifacts comprises means for generating one or more templates selected from the group consisting of Terraform, AWS CloudFormation, Pulumi, or Ansible templates.

Example 41 includes the subject matter of any of Examples 37-40, and wherein the simulation components are deployed as one or more of serverless functions, containers, or virtual machines in the cloud environment.

Example 42 includes the subject matter of any of Examples 37-41, and wherein the process diagram comprises a plurality of process modules selected from the group consisting of reactors, compressors, heat exchangers, separators, mixers, membranes, pumps valves, filters, distillation columns, absorption columns, extraction units, centrifuges, decanters, crystallizers, evaporators, condensors, and other chemical engineering modules.

Example 43 includes the subject matter of any of Examples 37-42, and wherein the GUI enables a user to specify parameters for each process module input flow, including at least one of flow rate, pressure, temperature, or composition.

Example 44 includes the subject matter of any of Examples 37-43, and wherein the structured graph model encodes a topology, parameters, and interconnections of the chemical process simulation.

Example 45 includes the subject matter of any of Examples 37-44, and wherein the simulation components communicate via message queues or events.

Example 46 includes the subject matter of any of Examples 37-45, and further including means for validating the structured graph model for logical consistency, completeness, or compliance with domain-specific rules prior to converting into IaC artifacts.

Example 47 includes the subject matter of any of Examples 37-46, and further including means for employing symbolic components working together with a large language model (LLM) to interpret the process diagram and assist in generating the structured graph model.

Example 48 includes the subject matter of any of Examples 37-47, and further including means for using an AI agent to validate the process diagram, detect errors, or suggest corrections prior to deployment.

Example 49 includes the subject matter of any of Examples 37-48, and wherein the conversion of the structured graph model into IaC artifacts is at least partially automated by a large language model and a neuro-symbolic reasoning engine.

Example 50 includes the subject matter of any of Examples 37-49, and further including means for employing an AI agent to select or configure mathematical or computational models for one or more process modules based on context or user goals.

Example 51 includes the subject matter of any of Examples 37-50, and further including means for using a neuro-symbolic system to analyze simulation results and iteratively refine process parameters or configurations in a feedback-driven optimization loop.

Example 52 includes the subject matter of any of Examples 37-51, and further including means for using an AI agent to translate between different modalities, including graphical user interface representations, structured files, and infrastructure-as-code artifacts.

Example 53 includes the subject matter of any of Examples 37-52, and further including means for maintaining a traceability record of simulation decisions, configurations, and execution outcomes for auditability and explainability.

Example 54 includes the subject matter of any of Examples 37-53, and further including means for employing a large language model (LLM) to generate a chemical process configuration in response to a specified production goal for a chemical material, wherein the LLM autonomously creates a process diagram and associated parameters suitable for achieving the production goal prior to converting the process configuration into simulation infrastructure.

Example 55 includes one or more non-transitory computer-readable media comprising a plurality of instructions stored thereon that, when executed, causes a compute device to provide a graphical user interface (GUI) for constructing and configuring a chemical process simulation; transform a process diagram created in the GUI into a structured graph model representing the process diagram; convert the structured graph model into infrastructure-as-code (IaC) artifacts for deployment in a cloud environment; and execute the IaC artifacts to provision and deploy simulation components in the cloud environment.

Example 56 includes the subject matter of Example 55, and wherein the plurality of instructions further cause the compute device to provide one or more simulation results to a user on the GUI.

Example 57 includes the subject matter of any of Examples 55 and 56, and wherein the structured graph model comprises a machine-readable representation selected from the group consisting of XML, JSON, or a domain-specific schema.

Example 58 includes the subject matter of any of Examples 55-57, and wherein to convert the structured graph model into infrastructure-as-code (IaC) artifacts comprises to generate one or more templates selected from the group consisting of Terraform, AWS CloudFormation, Pulumi, or Ansible templates.

Example 59 includes the subject matter of any of Examples 55-58, and wherein the simulation components are deployed as one or more of serverless functions, containers, or virtual machines in the cloud environment.

Example 60 includes the subject matter of any of Examples 55-59, and wherein the process diagram comprises a plurality of process modules selected from the group consisting of reactors, compressors, heat exchangers, separators, mixers, membranes, pumps valves, filters, distillation columns, absorption columns, extraction units, centrifuges, decanters, crystallizers, evaporators, condensors, and other chemical engineering modules.

Example 61 includes the subject matter of any of Examples 55-60, and wherein the GUI enables a user to specify parameters for each process module input flow, including at least one of flow rate, pressure, temperature, or composition.

Example 62 includes the subject matter of any of Examples 55-61, and wherein the structured graph model encodes a topology, parameters, and interconnections of the chemical process simulation.

Example 63 includes the subject matter of any of Examples 55-62, and wherein the simulation components communicate via message queues or events.

Example 64 includes the subject matter of any of Examples 55-63, and wherein the plurality of instructions further cause the compute device to validate the structured graph model for logical consistency, completeness, or compliance with domain-specific rules prior to converting into IaC artifacts.

Example 65 includes the subject matter of any of Examples 55-64, and wherein the plurality of instructions further cause the compute device to employ symbolic components working together with a large language model (LLM) to interpret the process diagram and assist in generating the structured graph model.

Example 66 includes the subject matter of any of Examples 55-65, and wherein the plurality of instructions further cause the compute device to use an AI agent to validate the process diagram, detect errors, or suggest corrections prior to deployment.

Example 67 includes the subject matter of any of Examples 55-66, and wherein the conversion of the structured graph model into IaC artifacts is at least partially automated by a large language model and a neuro-symbolic reasoning engine.

Example 68 includes the subject matter of any of Examples 55-67, and wherein the plurality of instructions further cause the compute device to employ an AI agent to select or configure mathematical or computational models for one or more process modules based on context or user goals.

Example 69 includes the subject matter of any of Examples 55-68, and wherein the plurality of instructions further cause the compute device to use a neuro-symbolic system to analyze simulation results and iteratively refine process parameters or configurations in a feedback-driven optimization loop.

Example 70 includes the subject matter of any of Examples 55-69, and wherein the plurality of instructions further cause the compute device to use an AI agent to translate between different modalities, including graphical user interface representations, structured files, and infrastructure-as-code artifacts.

Example 71 includes the subject matter of any of Examples 55-70, and wherein the plurality of instructions further cause the compute device to maintain a traceability record of simulation decisions, configurations, and execution outcomes for auditability and explainability.

Example 72 includes the subject matter of any of Examples 55-71, and wherein the plurality of instructions further cause the compute device to employ a large language model (LLM) to generate a chemical process configuration in response to a specified production goal for a chemical material, wherein the LLM autonomously creates a process diagram and associated parameters suitable for achieving the production goal prior to converting the process configuration into simulation infrastructure.

Example 73 includes a compute device comprising a processor; memory coupled to the processor; one or more computer readable media comprising a plurality of instructions stored thereon that, when executed by the processor, cause the processor to receive a process goal and one or more constraints for a chemical process; interpret the process goal and constraints to generate process requirements; generate a process diagram configured to achieve the process goal within the one or more constraints; and simulate the process diagram.

Example 74 includes the subject matter of Example 73, and wherein the plurality of instructions further cause the compute device to provide a user interface for specifying the process goal and one or more constraints.

Example 75 includes the subject matter of any of Examples 73 and 74, and wherein the user interface is configured to accept the process goal and constraints in at least one of a graphical, structured, or natural language format.

Example 76 includes the subject matter of any of Examples 73-75, and wherein the plurality of instructions further cause the compute device to generate a plurality of candidate process diagrams configured to achieve the process goal within the constraints.

Example 77 includes the subject matter of any of Examples 73-76, and wherein the plurality of instructions further cause the compute device to select and configuring process modules and parameters for each candidate process diagram.

Example 78 includes the subject matter of any of Examples 73-77, and wherein the plurality of instructions further cause the compute device to validate each candidate process configuration for logical consistency and feasibility.

Example 79 includes the subject matter of any of Examples 73-78, and wherein the plurality of instructions further cause the compute device to simulate each validated process configuration.

Example 80 includes the subject matter of any of Examples 73-79, and wherein the plurality of instructions further cause the compute device to compare simulation results to the process goal and constraints.

Example 81 includes the subject matter of any of Examples 73-80, and wherein the plurality of instructions further cause the compute device to iteratively refine the process diagram based on a comparison of the simulation results to the process goal and constraints until the process goal and constraints are satisfied.

Example 82 includes the subject matter of any of Examples 73-81, and wherein the process diagram comprises a plurality of process modules selected from the group consisting of reactors, compressors, heat exchangers, separators, mixers, and membranes.

Example 83 includes the subject matter of any of Examples 73-82, and wherein the process diagram is configured by specifying parameters including at least one of flow rate, pressure, temperature, or composition.

Example 84 includes the subject matter of any of Examples 73-83, and wherein the process goal comprises at least one of a target product specification, a performance metric, or an operational objective.

Example 85 includes the subject matter of any of Examples 73-84, and wherein the constraints comprise at least one of physical, safety, regulatory, or economic constraints.

Example 86 includes the subject matter of any of Examples 73-85, and wherein the process diagram is represented in a machine-readable format selected from the group consisting of XML, JSON, or a domain-specific schema.

Example 87 includes the subject matter of any of Examples 73-86, and wherein to simulate the process diagram comprises to deploy simulation components as one or more of serverless functions, containers, or virtual machines in a cloud environment.

Example 88 includes the subject matter of any of Examples 73-87, and wherein the plurality of instructions further cause the compute device to convert the process diagram into infrastructure-as-code (IaC) artifacts for deployment in a cloud environment.

Example 89 includes the subject matter of any of Examples 73-88, and wherein the plurality of instructions further cause the compute device to provide simulation results and feedback to a user via a user interface.

Example 90 includes the subject matter of any of Examples 73-89, and wherein the plurality of instructions further cause the compute device to employ a large language model (LLM) or neuro-symbolic AI to interpret the process goal, constraints, or process diagram.

Example 91 includes the subject matter of any of Examples 73-90, and wherein the plurality of instructions further cause the compute device to use an AI agent to generate candidate process diagrams based on the process goal and constraints.

Example 92 includes the subject matter of any of Examples 73-91, and wherein the plurality of instructions further cause the compute device to use an AI agent to select or configure mathematical or computational models for one or more process modules based on context or user goals.

Example 93 includes the subject matter of any of Examples 73-92, and wherein the plurality of instructions further cause the compute device to employ a neuro-symbolic system to validate process configurations for logical consistency, completeness, or compliance with domain-specific rules.

Example 94 includes the subject matter of any of Examples 73-93, and wherein the plurality of instructions further cause the compute device to use a neuro-symbolic system to analyze simulation results and iteratively refine process parameters or configurations in a feedback-driven optimization loop.

Example 95 includes the subject matter of any of Examples 73-94, and wherein the plurality of instructions further cause the compute device to use an AI agent to translate between different modalities, including graphical user interface representations, structured files, and infrastructure-as-code artifacts.

Example 96 includes the subject matter of any of Examples 73-95, and wherein the plurality of instructions further cause the compute device to maintain a traceability record of simulation decisions, configurations, and execution outcomes for auditability and explainability.

Example 97 includes a method comprising receiving, by a compute device, a process goal and one or more constraints for a chemical process; interpreting, by the compute device, the process goal and constraints to generate process requirements; generating, by the compute device, a process diagram configured to achieve the process goal within the one or more constraints; and simulating, by the compute device, the process diagram.

Example 98 includes the subject matter of Example 97, and further including providing, by the compute device, a user interface for specifying the process goal and one or more constraints.

Example 99 includes the subject matter of any of Examples 97 and 98, and wherein the user interface is configured to accept the process goal and constraints in at least one of a graphical, structured, or natural language format.

Example 100 includes the subject matter of any of Examples 97-99, and further including generating, by the compute device, a plurality of candidate process diagrams configured to achieve the process goal within the constraints.

Example 101 includes the subject matter of any of Examples 97-100, and further including selecting and configuring, by the compute device, process modules and parameters for each candidate process diagram.

Example 102 includes the subject matter of any of Examples 97-101, and further including validating, by the compute device, each candidate process configuration for logical consistency and feasibility.

Example 103 includes the subject matter of any of Examples 97-102, and further including simulating, by the compute device, each validated process configuration.

Example 104 includes the subject matter of any of Examples 97-103, and further including comparing, by the compute device, simulation results to the process goal and constraints.

Example 105 includes the subject matter of any of Examples 97-104, and further including iteratively refining, by the compute device, the process diagram based on a comparison of the simulation results to the process goal and constraints until the process goal and constraints are satisfied.

Example 106 includes the subject matter of any of Examples 97-105, and wherein the process diagram comprises a plurality of process modules selected from the group consisting of reactors, compressors, heat exchangers, separators, mixers, and membranes.

Example 107 includes the subject matter of any of Examples 97-106, and wherein the process diagram is configured by specifying parameters including at least one of flow rate, pressure, temperature, or composition.

Example 108 includes the subject matter of any of Examples 97-107, and wherein the process goal comprises at least one of a target product specification, a performance metric, or an operational objective.

Example 109 includes the subject matter of any of Examples 97-108, and wherein the constraints comprise at least one of physical, safety, regulatory, or economic constraints.

Example 110 includes the subject matter of any of Examples 97-109, and wherein the process diagram is represented in a machine-readable format selected from the group consisting of XML, JSON, or a domain-specific schema.

Example 111 includes the subject matter of any of Examples 97-110, and wherein simulating the process diagram comprises deploying simulation components as one or more of serverless functions, containers, or virtual machines in a cloud environment.

Example 112 includes the subject matter of any of Examples 97-111, and further including converting the process diagram into infrastructure-as-code (IaC) artifacts for deployment in a cloud environment.

Example 113 includes the subject matter of any of Examples 97-112, and further including providing simulation results and feedback to a user via a user interface.

Example 114 includes the subject matter of any of Examples 97-113, and further including employing a large language model (LLM) or neuro-symbolic AI to interpret the process goal, constraints, or process diagram.

Example 115 includes the subject matter of any of Examples 97-114, and further including using an AI agent to generate candidate process diagrams based on the process goal and constraints.

Example 116 includes the subject matter of any of Examples 97-115, and further including using an AI agent to select or configure mathematical or computational models for one or more process modules based on context or user goals.

Example 117 includes the subject matter of any of Examples 97-116, and further including employing a neuro-symbolic system to validate process configurations for logical consistency, completeness, or compliance with domain-specific rules.

Example 118 includes the subject matter of any of Examples 97-117, and further including using a neuro-symbolic system to analyze simulation results and iteratively refine process parameters or configurations in a feedback-driven optimization loop.

Example 119 includes the subject matter of any of Examples 97-118, and further including using an AI agent to translate between different modalities, including graphical user interface representations, structured files, and infrastructure-as-code artifacts.

Example 120 includes the subject matter of any of Examples 97-119, and further including maintaining a traceability record of simulation decisions, configurations, and execution outcomes for auditability and explainability.

Example 121 includes a compute device comprising means for receiving a process goal and one or more constraints for a chemical process; means for interpreting the process goal and constraints to generate process requirements; means for generating a process diagram configured to achieve the process goal within the one or more constraints; and means for simulating the process diagram.

Example 122 includes the subject matter of Example 121, and further including means for providing a user interface for specifying the process goal and one or more constraints.

Example 123 includes the subject matter of any of Examples 121 and 122, and wherein the user interface is configured to accept the process goal and constraints in at least one of a graphical, structured, or natural language format.

Example 124 includes the subject matter of any of Examples 121-123, and further including means for generating a plurality of candidate process diagrams configured to achieve the process goal within the constraints.

Example 125 includes the subject matter of any of Examples 121-124, and further including means for selecting and configuring process modules and parameters for each candidate process diagram.

Example 126 includes the subject matter of any of Examples 121-125, and further including means for validating each candidate process configuration for logical consistency and feasibility.

Example 127 includes the subject matter of any of Examples 121-126, and further including means for simulating each validated process configuration.

Example 128 includes the subject matter of any of Examples 121-127, and further including means for comparing simulation results to the process goal and constraints.

Example 129 includes the subject matter of any of Examples 121-128, and further including iteratively refining the process diagram based on a comparison of the simulation results to the process goal and constraints until the process goal and constraints are satisfied.

Example 130 includes the subject matter of any of Examples 121-129, and wherein the process diagram comprises a plurality of process modules selected from the group consisting of reactors, compressors, heat exchangers, separators, mixers, and membranes.

Example 131 includes the subject matter of any of Examples 121-130, and wherein the process diagram is configured by specifying parameters including at least one of flow rate, pressure, temperature, or composition.

Example 132 includes the subject matter of any of Examples 121-131, and wherein the process goal comprises at least one of a target product specification, a performance metric, or an operational objective.

Example 133 includes the subject matter of any of Examples 121-132, and wherein the constraints comprise at least one of physical, safety, regulatory, or economic constraints.

Example 134 includes the subject matter of any of Examples 121-133, and wherein the process diagram is represented in a machine-readable format selected from the group consisting of XML, JSON, or a domain-specific schema.

Example 135 includes the subject matter of any of Examples 121-134, and wherein the means for simulating the process diagram comprises means for deploying simulation components as one or more of serverless functions, containers, or virtual machines in a cloud environment.

Example 136 includes the subject matter of any of Examples 121-135, and further including means for converting the process diagram into infrastructure-as-code (IaC) artifacts for deployment in a cloud environment.

Example 137 includes the subject matter of any of Examples 121-136, and further including means for providing simulation results and feedback to a user via a user interface.

Example 138 includes the subject matter of any of Examples 121-137, and further including means for employing a large language model (LLM) or neuro-symbolic AI to interpret the process goal, constraints, or process diagram.

Example 139 includes the subject matter of any of Examples 121-138, and further including means for using an AI agent to generate candidate process diagrams based on the process goal and constraints.

Example 140 includes the subject matter of any of Examples 121-139, and further including means for using an AI agent to select or configure mathematical or computational models for one or more process modules based on context or user goals.

Example 141 includes the subject matter of any of Examples 121-140, and further including means for employing a neuro-symbolic system to validate process configurations for logical consistency, completeness, or compliance with domain-specific rules.

Example 142 includes the subject matter of any of Examples 121-141, and further including means for using a neuro-symbolic system to analyze simulation results and iteratively refine process parameters or configurations in a feedback-driven optimization loop.

Example 143 includes the subject matter of any of Examples 121-142, and further including means for using an AI agent to translate between different modalities, including graphical user interface representations, structured files, and infrastructure-as-code artifacts.

Example 144 includes the subject matter of any of Examples 121-143, and further including means for maintaining a traceability record of simulation decisions, configurations, and execution outcomes for auditability and explainability.

Example 145 includes one or more non-transitory computer-readable media comprising a plurality of instructions stored thereon that, when executed, causes a compute device to receive a process goal and one or more constraints for a chemical process; interpret the process goal and constraints to generate process requirements; generate a process diagram configured to achieve the process goal within the one or more constraints; and simulate the process diagram.

Example 146 includes the subject matter of Example 145, and wherein the plurality of instructions further cause the compute device to provide a user interface for specifying the process goal and one or more constraints.

Example 147 includes the subject matter of any of Examples 145 and 146, and wherein the user interface is configured to accept the process goal and constraints in at least one of a graphical, structured, or natural language format.

Example 148 includes the subject matter of any of Examples 145-147, and wherein the plurality of instructions further cause the compute device to generate a plurality of candidate process diagrams configured to achieve the process goal within the constraints.

Example 149 includes the subject matter of any of Examples 145-148, and wherein the plurality of instructions further cause the compute device to select and configuring process modules and parameters for each candidate process diagram.

Example 150 includes the subject matter of any of Examples 145-149, and wherein the plurality of instructions further cause the compute device to validate each candidate process configuration for logical consistency and feasibility.

Example 151 includes the subject matter of any of Examples 145-150, and wherein the plurality of instructions further cause the compute device to simulate each validated process configuration.

Example 152 includes the subject matter of any of Examples 145-151, and wherein the plurality of instructions further cause the compute device to compare simulation results to the process goal and constraints.

Example 153 includes the subject matter of any of Examples 145-152, and wherein the plurality of instructions further cause the compute device to iteratively refine the process diagram based on a comparison of the simulation results to the process goal and constraints until the process goal and constraints are satisfied.

Example 154 includes the subject matter of any of Examples 145-153, and wherein the process diagram comprises a plurality of process modules selected from the group consisting of reactors, compressors, heat exchangers, separators, mixers, and membranes.

Example 155 includes the subject matter of any of Examples 145-154, and wherein the process diagram is configured by specifying parameters including at least one of flow rate, pressure, temperature, or composition.

Example 156 includes the subject matter of any of Examples 145-155, and wherein the process goal comprises at least one of a target product specification, a performance metric, or an operational objective.

Example 157 includes the subject matter of any of Examples 145-156, and wherein the constraints comprise at least one of physical, safety, regulatory, or economic constraints.

Example 158 includes the subject matter of any of Examples 145-157, and wherein the process diagram is represented in a machine-readable format selected from the group consisting of XML, JSON, or a domain-specific schema.

Example 159 includes the subject matter of any of Examples 145-158, and wherein to simulate the process diagram comprises to deploy simulation components as one or more of serverless functions, containers, or virtual machines in a cloud environment.

Example 160 includes the subject matter of any of Examples 145-159, and wherein the plurality of instructions further cause the compute device to convert the process diagram into infrastructure-as-code (IaC) artifacts for deployment in a cloud environment.

Example 161 includes the subject matter of any of Examples 145-160, and wherein the plurality of instructions further cause the compute device to provide simulation results and feedback to a user via a user interface.

Example 162 includes the subject matter of any of Examples 145-161, and wherein the plurality of instructions further cause the compute device to employ a large language model (LLM) or neuro-symbolic AI to interpret the process goal, constraints, or process diagram.

Example 163 includes the subject matter of any of Examples 145-162, and wherein the plurality of instructions further cause the compute device to use an AI agent to generate candidate process diagrams based on the process goal and constraints.

Example 164 includes the subject matter of any of Examples 145-163, and wherein the plurality of instructions further cause the compute device to use an AI agent to select or configure mathematical or computational models for one or more process modules based on context or user goals.

Example 165 includes the subject matter of any of Examples 145-164, and wherein the plurality of instructions further cause the compute device to employ a neuro-symbolic system to validate process configurations for logical consistency, completeness, or compliance with domain-specific rules.

Example 166 includes the subject matter of any of Examples 145-165, and wherein the plurality of instructions further cause the compute device to use a neuro-symbolic system to analyze simulation results and iteratively refine process parameters or configurations in a feedback-driven optimization loop.

Example 167 includes the subject matter of any of Examples 145-166, and wherein the plurality of instructions further cause the compute device to use an AI agent to translate between different modalities, including graphical user interface representations, structured files, and infrastructure-as-code artifacts.

Example 168 includes the subject matter of any of Examples 145-167, and wherein the plurality of instructions further cause the compute device to maintain a traceability record of simulation decisions, configurations, and execution outcomes for auditability and explainability.

### Computer Program Listing

The following Terraform code provisions cloud infrastructure components—including S3 buckets, Lambda functions, an SQS queue, and an EC2 instance—that may correspond to the process modules and data flow illustrated in FIGS. 4 and 8. This configuration enables automated ingestion, processing, orchestration, and computation of chemical engineering simulation data in a cloud environment.

```
# main.tf
provider “aws” {
  region = var.aws_region
}
# S3 Buckets
resource “aws_s3_bucket” “excel_input_a” {
  bucket = var.bucket_a_name
  acl = “private”
  tags = {
    Name = “Excel Input A”
    Environment = “Production”
  }
}
resource “aws_s3_bucket” “excel_input_b” {
  bucket = var.bucket_b_name
  acl = “private”
  tags = {
    Name = “Excel Input B”
    Environment = “Production”
  }
}
# IAM Role for Lambda
resource “aws_iam_role” “lambda_role” {
  name = “lambda_execution_role”
  assume_role_policy = jsonencode({
    Version = “2012-10-17”
    Statement = [
      {
        Action = “sts:AssumeRole”
        Effect = “Allow”
        Principal = {
          Service = “lambda.amazonaws.com”
        }
      }
    ]
  })
}
# IAM Policy for Lambda
resource “aws_iam_role_policy” “lambda_policy” {
  name = “lambda_policy”
  role = aws_iam_role.lambda_role.id
  policy = jsonencode({
    Version = “2012-10-17”
    Statement = [
      {
        Action = [
          “s3:GetObject”,
          “s3:PutObject”,
          “logs:CreateLogGroup”,
          “logs:CreateLogStream”,
          “logs:PutLogEvents”,
          “sqs:SendMessage”,
          “sqs:ReceiveMessage”
        ]
        Effect = “Allow”
        Resource = [
          aws_s3_bucket.excel_input_a.arn,
          aws_s3_bucket.excel_input_b.arn,
          “${aws_sqs_queue.process_queue.arn}*”
        ]
      }
    ]
  })
}
# Lambda Functions
resource “aws_lambda_function” “compressor” {
  filename = “compressor.zip”
  function_name = “compressor”
  role = aws_iam_role.lambda_role.arn
  handler = “index.handler”
  runtime = “python3.12”
  environment {
    variables = {
      SQS_QUEUE_URL = aws_sqs_queue.process_queue.id
    }
  }
  tags = {
    Name = “Compressor Lambda”
  }
}
resource “aws_lambda_function” “mixer” {
  filename = “mixer.zip”
  function_name = “mixer”
  role = aws_iam_role.lambda_role.arn
  handler = “index.handler”
  runtime = “python3.12”
  environment {
    variables = {
      SQS_QUEUE_URL = aws_sqs_queue.process_queue.id
    }
  }
  tags = {
    Name = “Mixer Lambda”
  }
}
# S3 Event Notifications for Lambda
resource “aws_lambda_permission” “allow_bucket_a” {
  statement_id = “AllowExecutionFromS3BucketA”
  action = “lambda:InvokeFunction”
  function_name = aws_lambda_function.compressor.arn
  principal = “s3.amazonaws.com”
  source_arn = aws_s3_bucket.excel_input_a.arn
}
resource “aws_lambda_permission” “allow_bucket_b” {
  statement_id = “AllowExecutionFromS3BucketB”
  action = “lambda:InvokeFunction”
  function_name = aws_lambda_function.mixer.arn
  principal = “s3.amazonaws.com”
  source_arn = aws_s3_bucket.excel_input_b.arn
}
resource “aws_s3_bucket_notification” “bucket_a_notification” {
  bucket = aws_s3_bucket.excel_input_a.id
  lambda_function {
    lambda_function_arn = aws_lambda_function.compressor.arn
    events = [“s3:ObjectCreated:*”]
  }
  depends_on = [aws_lambda_permission.allow_bucket_a]
}
resource “aws_s3_bucket_notification” “bucket_b_notification” {
  bucket = aws_s3_bucket.excel_input_b.id
  lambda_function {
    lambda_function_arn = aws_lambda_function.mixer.arn
    events = [“s3:ObjectCreated:*”]
  }
  depends_on = [aws_lambda_permission.allow_bucket_b]
}
# SQS Queue
resource “aws_sqs_queue” “process_queue” {
  name = “process-queue”
  delay_seconds = 0
  max_message_size = 262144
  message_retention_seconds = 86400
  receive_wait_time_seconds = 10
  tags = {
    Environment = “production”
  }
}
# EC2 Instance
resource “aws_instance” “compute_instance” {
  ami = “ami-0c02fb55956c7d316” # Amazon Linux 2 AMI
  instance_type = var.instance_type
  iam_instance_profile = aws_iam_instance_profile.ec2_profile.name
  user_data = <<−EOF
            #!/bin/bash
            yum update −y
            yum install −y python3 python3-pip
            pip3 install boto3
            EOF
  tags = {
    Name = “ComputeInstance”
  }
}
# IAM Role for EC2
resource “aws_iam_role” “ec2_role” {
  name = “ec2_sqs_role”
  assume_role_policy = jsonencode({
    Version = “2012-10-17”
    Statement = [
      {
        Action = “sts:AssumeRole”
        Effect = “Allow”
        Principal = {
          Service = “ec2.amazonaws.com”
        }
      }
    ]
  })
}
resource “aws_iam_role_policy” “ec2_policy” {
  name = “ec2_sqs_policy”
  role = aws_iam_role.ec2_role.id
  policy = jsonencode({
    Version = “2012-10-17”
    Statement = [
      {
        Action = [
          “sqs:ReceiveMessage”,
          “sqs:DeleteMessage”,
          “sqs:GetQueueAttributes”
        ]
        Effect = “Allow”
        Resource = aws_sqs_queue.process_queue.arn
      }
    ]
  })
}
resource “aws_iam_instance_profile” “ec2_profile” {
  name = “ec2_profile”
  role = aws_iam_role.ec2_role.name
}
# Neuro-Symbolic AI Components
resource “aws_lambda_function” “neural_network” {
  filename = “neural_network.zip”
  function_name = “neural_network_module”
  role = aws_iam_role.lambda_role.arn
  handler = “index.handler”
  runtime = “python3.12”
  timeout = 300
  environment {
    variables = {
      S3_BUCKET_A = aws_s3_bucket.excel_input_a.bucket
      S3_BUCKET_B = aws_s3_bucket.excel_input_b.bucket
    }
  }
  tags = {
    Name = “Neural Network Lambda”
  }
}
resource “aws_lambda_function” “symbolic_reasoning” {
  filename = “symbolic_reasoning.zip”
  function_name = “symbolic_reasoning_engine”
  role = aws_iam_role.lambda_role.arn
  handler = “index.handler”
  runtime = “python3.12”
  timeout = 300
  tags = {
    Name = “Symbolic Reasoning Lambda”
  }
}
# Outputs
output “s3_bucket_a_name” {
  value = aws_s3_bucket.excel_input_a.bucket
}
output “s3_bucket_b_name” {
  value = aws_s3_bucket.excel_input_b.bucket
}
output “sqs_queue_url” {
  value = aws_sqs_queue.process_queue.id
}
output “ec2_instance_public_ip” {
  value = aws_instance.compute_instance.public_ip
}
```

1. A compute device for automating chemical engineering simulations, the compute device comprising:
a processor;
memory coupled to the processor;
one or more computer readable media comprising a plurality of instructions stored thereon that, when executed by the processor, cause the processor to:
provide a graphical user interface (GUI) for constructing and configuring a chemical process simulation;
transform a process diagram created in the GUI into a structured graph model representing the process diagram;
convert the structured graph model into infrastructure-as-code (IaC) artifacts for deployment in a cloud environment; and
execute the IaC artifacts to provision and deploy simulation components in the cloud environment.

2. The compute device of claim 1, wherein the plurality of instructions further cause the compute device to provide one or more simulation results to a user on the GUI.

3. The compute device of claim 1, wherein the structured graph model comprises a machine-readable representation selected from the group consisting of XML, JSON, or a domain-specific schema.

4. The compute device of claim 1, wherein to convert the structured graph model into infrastructure-as-code (IaC) artifacts comprises to generate one or more templates selected from the group consisting of Terraform, AWS CloudFormation, Pulumi, or Ansible templates.

5. The compute device of claim 1, wherein the process diagram comprises a plurality of process modules selected from the group consisting of reactors, compressors, heat exchangers, separators, mixers, membranes, pumps valves, filters, distillation columns, absorption columns, extraction units, centrifuges, decanters, crystallizers, evaporators, condensors, and other chemical engineering modules.

6. The compute device of claim 1, wherein the GUI enables a user to specify parameters for each process module input flow, including at least one of: flow rate, pressure, temperature, or composition.

7. The compute device of claim 1, wherein the structured graph model encodes a topology, parameters, and interconnections of the chemical process simulation.

8. The compute device of claim 1, wherein the simulation components communicate via message queues or events.

9. The compute device of claim 1, wherein the plurality of instructions further cause the compute device to validate the structured graph model for logical consistency, completeness, or compliance with domain-specific rules prior to converting into IaC artifacts.

10. The compute device of claim 1, wherein the plurality of instructions further cause the compute device to employ symbolic components working together with a large language model (LLM) to interpret the process diagram and assist in generating the structured graph model.

11. The compute device of claim 1, wherein the plurality of instructions further cause the compute device to use an AI agent to validate the process diagram, detect errors, or suggest corrections prior to deployment.

12. One or more non-transitory computer-readable media comprising a plurality of instructions stored thereon that, when executed, causes a compute device to:
provide a graphical user interface (GUI) for constructing and configuring a chemical process simulation;
transform a process diagram created in the GUI into a structured graph model representing the process diagram;
convert the structured graph model into infrastructure-as-code (IaC) artifacts for deployment in a cloud environment; and
execute the IaC artifacts to provision and deploy simulation components in the cloud environment.

13. The one or more non-transitory computer-readable media of claim 12, wherein the conversion of the structured graph model into IaC artifacts is at least partially automated by a large language model and a neuro-symbolic reasoning engine.

14. The one or more non-transitory computer-readable media of claim 12, wherein the plurality of instructions further cause the compute device to employ an AI agent to select or configure mathematical or computational models for one or more process modules based on context or user goals.

15. The one or more non-transitory computer-readable media of claim 12, wherein the plurality of instructions further cause the compute device to use a neuro-symbolic system to analyze simulation results and iteratively refine process parameters or configurations in a feedback-driven optimization loop.

16. A compute device comprising:
a processor;
memory coupled to the processor;
one or more computer readable media comprising a plurality of instructions stored thereon that, when executed by the processor, cause the processor to:
receive a process goal and one or more constraints for a chemical process;
interpret the process goal and constraints to generate process requirements;
generate a process diagram configured to achieve the process goal within the one or more constraints; and
simulate the process diagram.

17. The compute device of claim 16, wherein the plurality of instructions further cause the compute device to provide a user interface for specifying the process goal and one or more constraints.

18. The compute device of claim 17, wherein the user interface is configured to accept the process goal and constraints in at least one of: a graphical, structured, or natural language format.

19. The compute device of claim 16, wherein the plurality of instructions further cause the compute device to generate a plurality of candidate process diagrams configured to achieve the process goal within the constraints.

20. The compute device of claim 19, wherein the plurality of instructions further cause the compute device to select and configuring process modules and parameters for each candidate process diagram.
